(19) United States
(12) Patent Application Publication
(10) Pub. No.: US 2014/0223588 A1
(43) Pub. Date: Aug. 7, 2014

(54) A2M FRAGMENTS AND APPLICATIONS THEREOF

(71) Applicants: Isabelle Catherine Batxelli, Aigues-Vives (FR); Bénédicte Watelet, Saint Clement de Riviere (FR)

(72) Inventors: Isabelle Catherine Batxelli, Aigues-Vives (FR); Bénédicte Watelet, Saint Clement de Riviere (FR)

(73) Assignee: BIO-RAD INNOVATIONS, Marnes La Coquette (FR)

(21) Appl. No.: 14/234,503

(22) PCT Filed: Jul. 27, 2011
(86) PCT No.: PCT/IB2011/002066

§ 371 Date: Mar. 31, 2014

(51) Int. Cl. C07K 14/47; C07K 16/18; C07K 7/08; C07K 7/06

(52) U.S. Cl. CPC C07K 14/4717; C07K 7/06; C07K 16/18; C07K 7/08
USPC 800/8; 530/380; 530/328; 536/23.5; 435/320.1; 530/387.9; 436/501; 424/139.1; 530/327; 530/326; 435/252.33; 435/252.3; 435/254.2; 435/325; 435/419; 435/254.21

(57) ABSTRACT

The application relates to a polypeptide, the amino acid sequence of which is the sequence of a sub-fragment of the C-terminal thioester-cleaved fragment of human alpha-2-macroglobulin (A2M), wherein the molecular weight of said polypeptide is of 36 to 44 kDa, and wherein the first N-terminal amino acid of said polypeptide is an amino acid, which, in the full length sequence of said human A2M, is at a position 1,098 or 1,085 or 1,084 or 1,083, and the last C-terminal amino acid of said polypeptide is an amino acid, which, in the full length sequence of said human A2M, is one of the last twenty C-terminal amino acids. This polypeptide is differently abundant depending on the stage of liver fibrosis. The application also relates to means deriving therefrom and to the application thereof, notably in the field of hepatitis.

### FIELD OF THE INVENTION

The application relates to alpha-2-macroglobulin (A2M) polypeptides, the amino acid sequence of which is the sequence of A2M fragments, as well as to means deriving therefrom, such as ligands, which specifically bind to said A2M fragments, and peptides that can be used in the production of such fragments. The application also relates to medical and biological applications of said A2M fragments and of said means, more particularly in the field of hepatitis, still more particularly for the staging of hepatic fibrosis.

### BACKGROUND OF THE INVENTION

Hepatitis C virus (HCV) is a major cause of chronic liver disease and consequently a significant cause of morbidity and mortality. Indeed, about 180 million people are infected worldwide and up to 70% of HCV-positive patients will develop chronic infection. Chronically infected patients are at severe risk of developing hepatic fibrosis and cirrhosis. Hepatic fibrosis is mediated by necro-inflammation and activation of liver stellate cells; it corresponds to an excess accumulation of extracellular matrix proteins including collagen. Fibrosis progression in chronic hepatitis C determines the final prognosis and thus the need of treatment. In chronic hepatitis C, the transition from mild to moderate fibrosis seems to be a major prognostic step.

Currently, liver biopsy is considered the gold standard for the diagnosis/staging of hepatic fibrosis, but this invasive method is now being progressively replaced or combined with non-invasive methods, such as blood tests or hepatic elasticity measurement [Ahmad et al. 2011].

Various serum molecules have been assessed as potential markers of the fibrosis stage, and many different marker combinations include such molecules.

For example:
FIBROSPECT® is a combination of hyaluronic acid (HA), metalloproteinase inhibitor 1 (TIMP-1) and alpha-2-macroglobulin (A2M) [Zaman et al. 2007];
FIBROTEST® is the combination of alpha-2-macroglobulin (A2M), haptoglobin (Hapto), apolipoprotein A1 (ApoA1), gamma glutamyl transpeptidase (GGT) and total bilirubin (BLT) [Imbert-Bismut et al. 2001; Poynard T et al. 2004];
FIBROMETER™ is a combination of hyaluronic acid (HA), aspartate aminotransferase (AST), platelet count (PLQ), prothrombin index (TP), alpha-2-macroglobuline (A2M), urea and age [Cales et al. 2008 and 2010]
HEPASCORE™ is a combination of bilirubin (BLT), gamma glutamyl transpeptidase (GGT), hyaluronic acid (HA), alpha-2-macroglobulin (A2M), gender and age [Guechot et al. 2010].

Many other markers and marker combinations have been described, such as:
the PGAA index, which combines gamma glutamyl transpeptidase (GGT), apolipoprotein A1 (ApoA1), prothrombin index (TP) and alpha-2-macroglobulin (A2M) [Naveau et al. 1994];
ACTITEST™, which is a modified form of the FIBROTEST® with the addition of ALT level [Imbert-Bismut et al. 2001];
the marker combination, which has been described in the patent applications FR 1151022 and U.S. 61/440,968 filed on 9 Feb. 2011, which notably involves secreted phosphoprotein 1 (SPP1), alpha-2-macroglobulin (A2M) and/or vimentin (VIM), interleukin-8 (IL8) and/or C—X—C motif chemokine 10 (CXCL10) and/or endoglin (ENG).

The search for a marker combination with the required precision and accuracy, more particularly at the early stage of fibrosis, fosters the search for new serum markers having an improved prognostic value [Shaheen et al. 2007; Ahmad et al. 2011].

### SUMMARY OF THE INVENTION

The present application relates to the subject-matter as defined in the claims as filed and as herein described.

The application provides polypeptides, the sequence of which is the sequence of fragments of alpha-2-macroglobulin (A2M). These A2M polypeptides have been isolated from the sera of hepatitis patients, and which are differentially abundant depending on the stage of hepatic fibrosis. Remarkably, these A2M polypeptides are more abundant in those hepatitis patients, whose liver fibrosis scores at least F2, than in those patients, whose liver fibrosis scores at most F1 according to the Metavir scoring system.

The application relates to these A2M polypeptides, as well as to means deriving therefrom, such as:
ligands, which specifically bind to said A2M polypeptides, more particularly antibodies and antibody fragments,
peptides, which are fragments of said A2M polypeptides, and which can be used in the production of said A2M antibodies and antibody fragments,
compounds, which comprise at least one A2M polypeptide or peptide of the application,
nucleic acids coding for said A2M polypeptides, vectors and host cells comprising at least one of said nucleic acids or at least one of said A2M polypeptides,
primers and probes, which specifically amplify such nucleic acids.

The application also relates to medical and biological applications of said A2M polypeptides and of said means, more particularly in the field of hepatitis, still more particularly for staging hepatic fibrosis, even still more particularly for determining whether a human liver is a fibrosis stage, which scores at most F1, or whether it scores at least F2 according to the Metavir scoring system.

### BRIEF DESCRIPTION OF THE FIGURES

In the figures, as well as throughout the application text:

α2M is equal to A2M, and means alpha-2-macroglobulin, more particularly human alpha-2-macroglobulin;

2-DE means two dimensional gel electrophoresis;

1-DE means one dimensional gel electrophoresis.

FIG. 1A: flow chart of the experimental approach.

FIG. 1B: comparative 2-DE analysis of F1 and F2 patients using a polyclonal anti-A2M antibody (60 or 120 micrograms of total proteins from the hepatitis C patient's serum [Metavir score F1 or F2], said serum having been previously depleted from albumin and from immunoglobulins G).

As expected, the full length monomeric A2M form (about 180 kDa), the thioester-cleaved N- and C-terminal A2M fragments (about 110 kDa and 56 kDa respectively) and the bait region-cleaved N- and C-terminal A2M fragments (about 75 kDa and about 85 kDa respectively) were clearly detected.

Furthermore, novel A2M fragments of about 40 kDa were detected as several distinct spots (“40 kDa region”). These novel A2M fragments of about 40 kDa are present at a significantly higher level in the F2 patients compared to the F1 patients.

FIGS. 2A, 2B and 2C: analysis of the A2M 40 kDa fragments.

FIG. 2A: nine 2-DE significant spots of A2M 40 kDa fragments permitting to discriminate F1 and F2 stages of fibrosis.

FIG. 2B: 2-DE spots of A2M 40 kDa fragments in nine F1 patients, ten F2 patients, one F3 patient and one F4 patient (Metavir staging).

FIG. 2C: 1-DE bands of A2M 40 kDa fragments in nine F1 patients and ten F2 patients (Metavir staging).

FIG. 3: box plots of spot intensity measurements versus Metavir fibrosis stage in the whole study population (HCV-infected patients with F1 fibrosis, n=9 and HCV-infected patients with F2 fibrosis, n=10; A2M40=A2M 40 kDa fragment).

FIGS. 4A, 4B and 4C: characterization of the A2M 40 kDa fragments.

FIG. 4A: detection of the different A2M fragments after 2-DE by western blot analysis using antibodies against the N-terminal (lower panel) and C-terminal (upper panel) regions of A2M.

FIG. 4B: schematic representation of the A2M monomer, of the N-terminal and C-terminal bait-cleaved A2M fragments, of the N-terminal and C-terminal thioester-cleaved A2M fragments, and of the A2M C-terminal fragments of about 40 kDa.

FIG. 4C: A2M C-terminal sequence (fragment 661-1,474; SEQ ID NO: 11): underlined amino acids correspond to the sequence of the A2M bait cleavage site (690-728) and of the thioester bond cleavage site (972-975); bold amino acids correspond to the sequence of some of the most significant peptides identified by the LC-MS/MS approach (peptides 1,083-1,092; 1,110-1,122; 1,148-1,163; 1,249-1,263; 1,374-1,384; 1,385-1,397; 1,432-1,440); the amino acid starting positions of the (four) A2M 40 kDa fragments are at positions 1,083 (Ser), 1,084 (Gly), 1,085 (Ser) and 1,098 (Glu), respectively.

FIGS. 5A and 5B: localization of the epitope of an anti-human C-terminal A2M rabbit polyclonal antibody (Abcam, ab58703) and of the epitope of an anti-human N-terminal A2M mouse monoclonal antibody (Abnova, H00000002-M03).

Overlapping synthetic peptides corresponding to the amino acid sequence of human A2M were prepared using the epitope mapping technique and probed with an anti-N-terminal A2M antibody (FIG. 5A) or an anti-C-terminal A2M antibody (FIG. 5B).

FIG. 6: N-terminal sequencing of the 40 kDa A2M fragments.

Visualization of the three bands corresponding to the three 40 kDa A2M fragments on a SDS-PAGE gel before excision for N-terminal sequencing (first lane: molecular weights; second and third lanes: 10 and 100 micrograms of A2M purified from human plasma, respectively). The huge quantity of A2M proteins analysed in this experience (100 micrograms) has influenced the protein migration in the gel and induced a localisation of the 40 kDa A2M fragments upper than 40 kDa. The three distinct bands were well-visualized after staining.

### DETAILED DESCRIPTION OF THE INVENTION

The application provides fragments of alpha-2-macroglobulin (A2M), which have been isolated from the sera of hepatitis patients. An A2M fragment of the application is a polypeptide, the amino acid sequence of which is the sequence of a C-terminal fragment of human alpha-2-macroglobulin (A2M), more particularly the sequence of a sub-fragment of the C-terminal thioester-cleaved fragment of human alpha-2-macroglobulin (A2M). The A2M fragments of the application are differentially abundant depending on the stage of hepatic fibrosis. Either individually or in combination with other marker(s) and/or clinical feature(s), they enable to reliably score the fibrosis stage. More particularly, they enable to differentiate no/mild fibrosis (Metavir score of at most F1) from moderate/advanced/severe fibrosis (Metavir score of F2 or more). They can be recombinantly produced, and are highly useful tools in the diagnosis, prognosis and treatment of hepatitis.

The application further provides sub-fragments of the A2M fragments of the application. The A2M sub-fragments of the application are N-terminal sub-fragments of the A2M fragments of the application. An A2M sub-fragment of the application is notably useful as an antigen for the production of antibodies and antibody fragments, which specifically bind to an A2M fragment of the application, and/or as an affinity ligand for screening for such antibodies or antibody fragments.

A polypeptide or peptide is a single linear chain of amino acids bonded together by peptide bonds. Said amino acid chain has an N-terminal extremity and a C-terminal extremity. In the application, peptides consist of at most 50 amino acids, whereas polypeptide consist of more than 50 amino acids. The A2M fragments of the application can be termed “polypeptides”, whereas the A2M sub-fragments of the application can be termed “peptides”. The term “A2M polypeptides of the application” (or the like) and the term “A2M fragment of the invention” (or the like) can be used interchangeably. Similarly, the term “A2M peptide of the application” (or the like) and the term “A2M sub-fragment of the invention” (or the like) can be used interchangeably.

In the application, unless specified otherwise and/or unless a context dictates otherwise, all the terms have their ordinary meaning in the relevant field(s).

The expression “fragment of human A2M” and the like is intended in accordance with its ordinary meaning in the field, and implies that the amino acid sequence of said fragment is identical to a continuous portion of the full length sequence of said human A2M from one amino acid to another, wherein at least one of said two amino acids is internal to said A2M full length sequence, and that said fragment is under an isolated form. The same applies to a sub-fragment of a fragment mutatis mutandis.

The application further provides compounds, nucleic acids, vectors, host cells, antibodies, antibody fragments, which derive from the A2M polypeptides of the application or from the A2M peptides of the application.

The application also encompasses applications of the means of the application, notably in the field of hepatitis, still more particularly for the staging of hepatic fibrosis.

A2M Polypeptides of the Application:

The A2M polypeptides of the application are A2M fragments, which have been identified and isolated by proteomic analysis of serum samples collected from HCV infected human patients. It is believed that, although human A2M is a well known protein, the A2M fragments of the application have never been described before.

Human A2M is a serum glycoprotein, which has sequence similarity with other members of the A2M family including the complement components C3, C4 and C5. Human A2M is synthesized by the liver (primarily by hepatocytes) as a polypeptide of usually 1,474 amino acids with a signal peptide (usually 23 residues). The mature A2M protein is a tetramer (about 720 kDa) of four identical 180 kDa subunits, which form two disulfide bond-linked dimers. A2M is a general and irreversible protease inhibitor implicated in many processes and can inhibit all four classes of proteases by a unique trapping mechanism.

A human A2M sequence is available e.g., under accession number NM_{—}000014, and is reproduced below:

| (full length human A2M; SEQ ID NO: 9) | |
|---|---|
| MGKNKLLHPS LVLLLLVLLP TDASVSGKPQ YMVLVPSLLH TETTEKGCVL LSYLNETVTV | 60 |
| SASLESVRGN RSLFTDLEAE NDVLHCVAFA VPKSSSNEEV MFLTVQVKGP TQEFKKRTTV | 120 |
| MVKNEDSLVF VQTDKSIYKP GQTVKFRVVS MDENFHPLNE LIPLVYIQDP KGNRIAQWQS | 180 |
| FQLEGGLKQF SFPLSSEPFQ GSYKVVVQKK SGGRTEHPFT VEEFVLPKFE VQVTVPKIIT | 240 |
| ILEEEMNVSV CGLYTYGKPV PGHVTVSICR KYSDASDCHG EDSQAFCEKF SGQLNSHGCF | 300 |
| YQQVKTKVFQ LKRKEYEMKL HTEAQIQEEG TIVELTGRQS SEITRTITKL SFVKVDSHFR | 360 |
| QGIPFFGQVR LVDGKGVPIP NKVIFIRGNE ANYYSNATTD EHGLVQFSIN TTNVMGTSLT | 420 |
| VRVNYKDRSP CYGYQWVSEE HEEAHHTAYL VFSPSKSFVH LEPMSHELPC GHTQTVQAHY | 480 |
| ILNGGTLLGL KKLSFYYLIM AKGGIVRTGT HGLLVKQEDM KGHFSISIPV KSDIAPVARL | 540 |
| LIYAVLPTGD VIGDSAKYDV ENCLANKVDL SFSPSQSLPA SHAHLRVTAA PQSVCALRAV | 600 |
| DQSVLLMKPD AELSASSVYN LLPEKDLTGF PGPLNDQDNE DCINRHNVYI NGITYTPVSS | 660 |
| TNEKDMYSFL EDMGLKAFTN SKIRKPKMCP QLQQYEMHGP EGLRVGFYES DVMGRGHARL | 720 |
| VHVEEPHTET VRKYFPETWI WDLVVVNSAG VAEVGVTVPD TITEWKAGAF CLSEDAGLGI | 780 |
| SSTASLRAFQ PFFVELTMPY SVIRGEAFTL KATVLNYLPK CIRVSVQLEA SPAFLAVPVE | 840 |
| KEQAPHCICA NGRQTVSWAV TPKSLGNVNF TVSAEALESQ ELCGTEVPSV PEHGRKDTVI | 900 |
| KPLLVEPEGL EKETTFNSLL CPSGGEVSEE LSLKLPPNVV EESARASVSV LGDILGSAMQ | 960 |
| NTQNLLQMPY GCGEQNMVLF APNIYVLDYL NETQQLTPEI KSKAIGYLNT GYQRQLNYKH | 1020 |
| YDGSYSTFGE RYGRNQGNTW LTAFVLKTFA QARAYIFIDE AHITQALIWL SQRQKDNGCF | 1080 |
| RSSGSLLNNA IKGGVEDEVT LSAYITIALL EIPLTVTHPV VRNALFCLES AWKTAQEGDH | 1140 |
| GSHVYTKALL AYAFALAGNQ DKRKEVLKSL NEEAVKKDNS VHWERPQKPK APVGHFYEPQ | 1200 |
| APSAEVEMTS YVLLAYLTAQ PAPTSEDLTS ATNIVKWITK QQNAQGGFSS TQDTVVALHA | 1260 |
| LSKYGAATFT RTGKAAQVTI QSSGTFSSKF QVDNNNRLLL QQVSLPELPG EYSMKVTGEG | 1320 |
| CVYLQTSLKY NILPEKEEFP FALGVQTLPQ TCDEPKAHTS FQISLSVSYT GSRSASNMAI | 1380 |
| VDVKMVSGFI PLKPTVKMLE RSNHVSRTEV SSNHVLIYLD KVSNQTLSLF FTVLQDVPVR | 1440 |
| DLKPAIVKVY DYYETDEFAI AEYNAPCSKD LGNA | 1474 |

In the application, residue or amino acid positions are computed with respect to a full length human A2M sequence of 1,474 amino acids, more particularly with respect to sequence NM_{—}000014 (SEQ ID NO: 9).

After cleavage of the 1-23 signal peptide, the A2M sequence of SEQ ID NO: 9 matures into:

| (SEQ ID NO: 10) | |
|---|---|
| SVSGKPQ YMVLVPSLLH TETTEKGCVL LSYLNETVTV | |
| SASLESVRGN RSLFTDLEAE NDVLHCVAFA VPKSSSNEEV MFLTVQVKGP TQEFKKRTTV | |
| MVKNEDSLVF VQTDKSIYKP GQTVKFRVVS MDENFHPLNE LIPLVYIQDP KGNRIAQWQS | |
| FQLEGGLKQF SFPLSSEPFQ GSYKVVVQKK SGGRTEHPFT VEEFVLPKFE VQVTVPKIIT | |
| ILEEEMNVSV CGLYTYGKPV PGHVTVSICR KYSDASDCHG EDSQAFCEKF SGQLNSHGCF | |
| YQQVKTKVFQ LKRKEYEMKL HTEAQIQEEG TVVELTGRQS SEITRTITKL SFVKVDSHFR | |
| QGIPFFGQVR LVDGKGVPIP NKVIFIRGNE ANYYSNATTD EHGLVQFSIN TTNVMGTSLT | |
| VRVNYKDRSP CYGYQWVSEE HEEAHHTAYL VFSPSKSFVH LEPMSHELPC GHTQTVQAHY | |
| ILNGGTLLGL KKLSFYYLIM AKGGIVRTGT HGLLVKQEDM KGHFSISIPV KSDIAPVARL | |
| LIYAVLPTGD VIGDSAKYDV ENCLANKVDL SFSPSQSLPA SHAHLRVTAA PQSVCALRAV | |
| DQSVLLMKPD AELSASSVYN LLPEKDLTGF PGPLNDQDNE DCINRHNVYI NGITYTPVSS | |
| TNEKDMYSFL EDMGLKAFTN SKIRKPKMCP QLQQYEMHGP EGLRVGFYES DVMGRGHARL | |
| VHVEEPHTET VRKYFPETWI WDLVVVNSAG VAEVGVTVPD TITEWKAGAF CLSEDAGLGI | |
| SSTASLRAFQ PFFVELTMPY SVIRGEAFTL KATVLNYLPK CIRVSVQLEA SPAFLAVPVE | |
| KEQAPHCICA NGRQTVSWAV TPKSLGNVNF TVSAEALESQ ELCGTEVPSV PEHGRKDTVI | |
| KPLLVEPEGL EKETTFNSLL CPSGGEVSEE LSLKLPPNVV EESARASVSV LGDILGSAMQ | |
| NTQNLLQMPY GCGEQNMVLF APNIYVLDYL NETQQLTPEI KSKAIGYLNT GYQRQLNYKH | |
| YDGSYSTFGE RYGRNQGNTW LTAFVLKTFA QARAYIFIDE AHITQALIWL SQRQKDNGCF | |
| RSSGSLLNNA IKGGVEDEVT LSAYITIALL EIPLTVTHPV VRNALFCLES AWKTAQEGDH | |
| GSHVYTKALL AYAFALAGNQ DKRKEVLKSL NEEAVKKDNS VHWERPQKPK APVGHFYEPQ | |
| APSAEVEMTS YVLLAYLTAQ PAPTSEDLTS ATNIVKWITK QQNAQGGFSS TQDTVVALHA | |
| LSKYGAATFT RTGKAAQVTI QSSGTFSSKF QVDNNNRLLL QQVSLPELPG EYSMKVTGEG | |
| CVYLQTSLKY NILPEKEEFP FALGVQTLPQ TCDEPKAHTS FQISLSVSYT GSRSASNMAI | |
| VDVKMVSGFI PLKPTVKMLE RSNHVSRTEV SSNHVLIYLD KVSNQTLSLF FTVLQDVPVR | |
| DLKPAIVKVY DYYETDEFAI AEYNAPCSKD LGNA | 1451 |

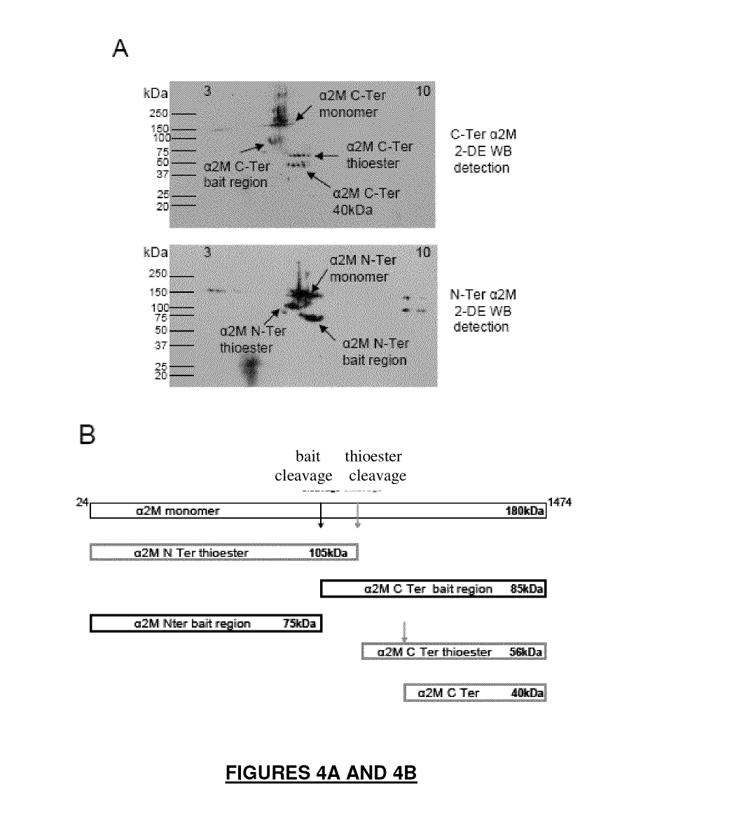

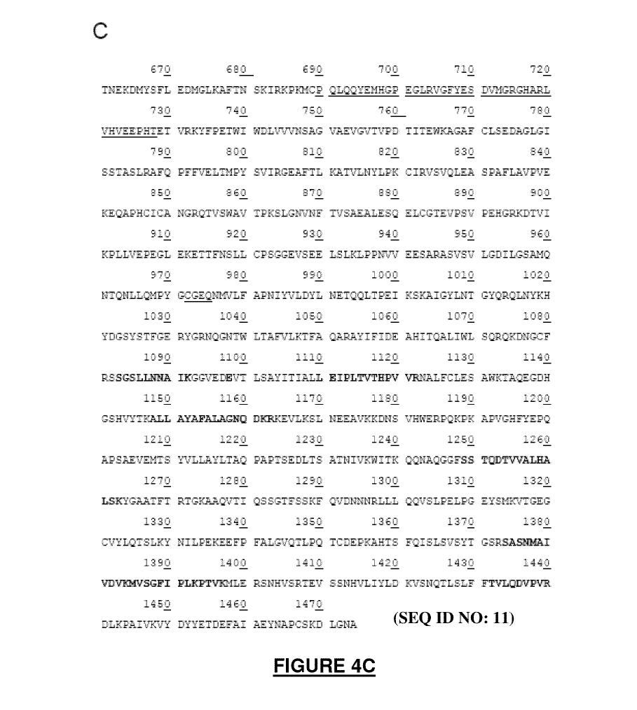

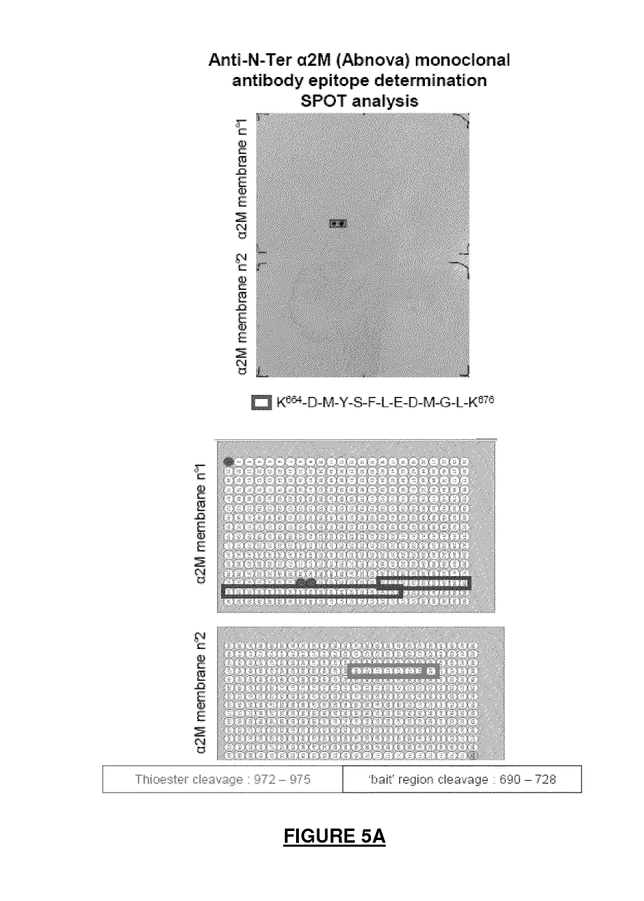

FIG. 4C shows the 661-1,474 C-terminal fragment (SEQ ID NO: 11) of said A2M sequence of SEQ ID NO: 9.

Naturally-occurring human A2M, the amino acid sequence of which differs from said sequence of SEQ ID NO: 9, have been described in the prior art. Examples of such naturally-occurring A2M variants include the proteins, the amino acid sequence of which differs from said sequence of SEQ ID NO: 9 by one or several of the following amino acid substitutions:
N639D (Asp at position 639 instead of Asn);
R704H (His at position 704 instead of Arg);
L815Q (Gln at position 815 instead of Leu);
C972Y (Tyr at position 972 instead of Cys);
I1000V (Val at position 1,000 instead of Ile).

The bait region of human A2M (residues 690-728 within SEQ ID NO: 9) contains specific cleavage sites for different proteases. Cleavage of the bait region by a protease induces a conformation change that enables A2M to trap and form a covalent bond with the protease. Specifically, following cleavage of the bait region, a thioester bond (residues 972-975 within SEQ ID NO: 9) is hydrolyzed to mediate the covalent binding of A2M to the protease. The trapped protease remains active against small peptide substrates, but loses its ability to interact with large protein substrates or inhibitors. A2M family members are major liver products associated with inflammatory response.

An A2M fragment of the application is a polypeptide, the amino acid sequence of which is the sequence of a C-terminal fragment of human alpha-2-macroglobulin (A2M), more particularly the sequence of a sub-fragment of the C-terminal bait-cleaved fragment of human A2M, still more particularly the sequence of a sub-fragment of the C-terminal thioester-cleaved fragment of human A2M. Please see FIG. 4B.

The site of said bait cleavage of said human A2M is at positions 690-728 within the sequence of the full length human A2M (e.g., within SEQ ID NO: 9 or a variant sequence thereof) and/or said C-terminal bait-cleaved fragment of said human A2M is of about 85 kDa.

The site of said thioester cleavage of said human A2M is at positions 972-975 within the sequence of the full length human A2M (e.g., within SEQ ID NO: 9 or a variant sequence thereof) and/or said C-terminal thioester-cleaved fragment of said human A2M is of about 56 kDa.

An A2M fragment of the application can notably be defined:
by feature(s) relating to the molecular weight of said A2M fragment, and/or
by feature(s) relating to the start and/or end position(s) of said fragment within said full length A2M protein, and/or
by feature(s) relating to the amino acid length of said fragment, and/or
by feature(s) relating to the location of said fragment within a full length A2M sequence, and/or
by feature(s) relating to the isoelectric point (pI) of said fragment.

The application explicitly encompasses each and every combination of such features. Hence, according to an aspect of the application, said A2M fragment is of about 40 kDa (cf. e.g., the examples below and the Figures). More precisely, according to said aspect of the application, said A2M fragment is of about 36 to about 44 kDa, more particularly of about 36 to 43.5 kDa, still more particularly of 36.5 to 43.5 kDa, still more particularly of about 38 to 43.5 kDa, still more particularly of 38.5 to 43.5 kDa, still more particularly of about 39 to 43.5 kDa, still more particularly of 39 to 43.5 kDa, still more particularly of about 40 to 43.5 kDa, still more particularly of 40.5 to 43.5 kDa, still more particularly of about 41 to 43.5 kDa, still more particularly of 41.5 to 43.5 kDa. A molecular weight (MW) can be determined by any conventional MW determination means that the person of ordinary skill in the art may find appropriate or convenient, e.g.,:
by addition of the molecular weights of the amino acids of said A2M fragment, e.g., following the method described in Gasteiger et al., 2005 and/or using a computer assisted tool, such as the ProtParam software (described in Gasteiger et al., 2005, and available from http://www.expasy.org/tools/protparam.html), and/or
by mass spectrometry, e.g., as described in the examples below, and/or
by one-dimensional electrophoresis or two-dimensional electrophoresis.

Said one-dimensional or two-dimensional electrophoresis may e.g., comprise a gel electrophoresis, more particularly a gel electrophoresis under denaturing conditions (e.g., under SDS denaturing conditions), e.g., under denaturing and reducing conditions, e.g., under the denaturing and reducing conditions described in the examples below (cf below, paragraph “One dimensional gel electrophoresis (1-DE)” and/or paragraph “Two dimensional gel electrophoresis (2-DE)”).

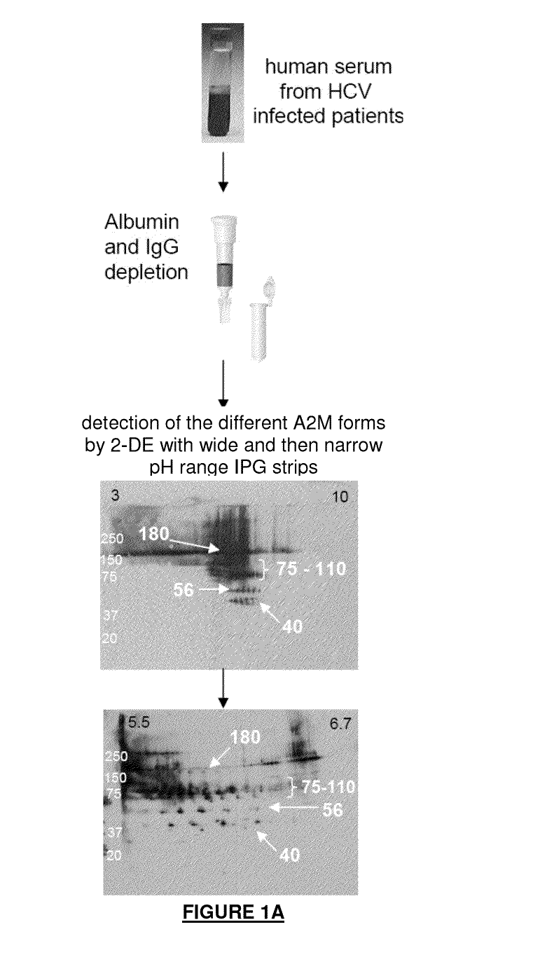

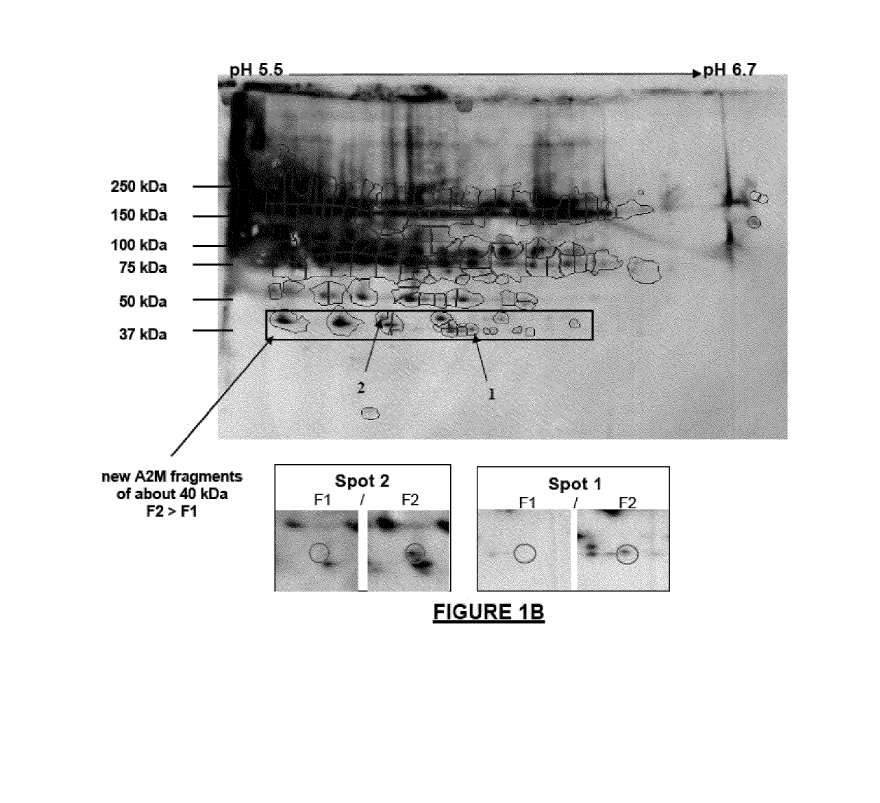

For said two-dimensional gel electrophoresis, pH conditions of 3 to 10, more particularly of 5.5 to 6.7 can e.g., be used (cf. below, paragraph “Two dimensional gel electrophoresis (2-DE)” and/or FIG. 1A and/or FIG. 1B).

Said two-dimensional electrophoresis may e.g., be as shown in, or illustrated by, FIG. 1A and/or in FIG. 1B.

The detection of the protein bands of said one-dimensional or two-dimensional gel electrophoresis can be performed by any means, which the person of ordinary skill in the art may find appropriate e.g.,:
by COOMASSIE® staining (e.g., COOMASSIE® Brilliant Blue R250) e.g., as described in the examples below (cf. below, paragraph “One dimensional gel electrophoresis (1-DE)”), or
by western blotting e.g., as described in the examples below (cf. below, paragraph” Two dimensional gel electrophoresis (2-DE)”).

The protein(s) submitted to said one-dimensional or two-dimensional gel electrophoresis may e.g., be:
purified human A2M (e.g., purified A2M that is commercially available from Sigma), or
at least one sample of serum of a hepatitis C patient [e.g., an HCV-infected patient scoring F1 or F2 according to the Metavir scoring scale], wherein said serum preferably has previously been depleted from albumin and immunoglobulins G (e.g., using the AURUM® kit, Bio-Rad, Hercules, U.S.A., cf. the examples below).

The amount of proteins submitted to one-dimensional gel electrophoresis may e.g., be:
when detection is performed by western blotting:
   0.5 to 5 micrograms of purified human A2M, or
   5 to 50 micrograms of the proteins of said albumin- and IgG-depleted serum;
when detection is performed by COOMASSIE® staining, 10 to 100 micrograms of purified human A2M.

The quantity of proteins submitted to two-dimensional gel electrophoresis may e.g., be:
when detection is performed by western blotting:
   1 to 10 micrograms of purified human A2M, or
   50 to 150 micrograms of the proteins of said albumin- and IgG-depleted serum;
when detection is performed by COOMASSIE® staining, 100 to 200 micrograms of purified human A2M.

According to an alternative and/or complementary aspect of the application:
the first N-terminal amino acid of said A2M fragment is an amino acid, which, in the full length sequence of said A2M, is at a position selected from positions 1,081-1,123, and/or, preferably and,
the last C-terminal amino acid of said A2M fragment is an amino acid, which, in the full length sequence of said A2M, is one of the last twenty C-terminal amino acids.

The expression “a position selected from positions X-Y” and the like explicitly encompasses position X and position Y as well as each and every position between X and Y. For example, “a position selected from positions 1,081-1,123” means position 1,081 or 1,082 or 1,083 or 1,084 or 1,085 or 1,086 or 1,087 or 1,088 or 1,089 or 1,090 or 1,091 or 1,092 or 1,093 or 1,094 or 1,095 or 1,096 or 1,097 or 1,098 or 1,099 or 1,100 or 1,101 or 1,102 or 1,103 or 1,104 or 1,105 or 1,106 or 1,107 or 1,108 or 1,109 or 1,110 or 1,111 or 1,112 or 1,113 or 1,114 or 1,115 or 1,116 or 1,117 or 1,118 or 1,119 or 1,120 or 1,121 or 1,122 or 1,123.

The expression “last twenty C-terminal amino acids” and the like means the stretch of twenty contiguous amino acids that are at the very C-terminal extremity of said A2M full length sequence. For example, if the full length sequence of said A2M consists of 1,474 amino acids, the last C-terminal amino acid of said fragment is an amino acid, which is at a position selected from positions 1,455-1,474 within said full length A2M sequence.

A human full length A2M sequence is considered to be consisting of 1,474 amino acids. Examples of such human full length A2M sequences comprise the above-described sequence of SEQ ID NO: 9 (NM_{—}000014) and the variant sequences thereof, which still are human A2M sequences. Such variants include the naturally-occurring human A2M variants, which are already known at the filing date of the present application, as well as any human A2M variant that may be described after said filing date. Such variant sequences usually differ from said sequence of SEQ ID NO: 9 by one or more amino acid deletions (e.g., amino acid deletion(s) from the C-terminal extremity and/or N-terminal extremity of SEQ ID NO: 9) and/or by one or more amino acid substitutions. More particularly, such variant sequences differ from said sequence of SEQ ID NO: 9 only by amino acid substitution(s). The total number of amino acid substitution(s) and deletion(s) does usually not exceed ten, more particularly eight, still more particularly five. Hence, said one or more amino acid substitutions usually are at most ten, more particularly at most eight, still more particularly at most five. Said amino acid substitution(s) usually comprise one or several of the above-mentioned (naturally-occurring) N639D, R704H, L815Q, C972Y, I1000V substitutions.

When said full length A2M sequence is the sequence of SEQ ID NO: 9, said first N-terminal amino acid of said A2M fragment is one of the following amino acids:

TABLE 4

| Position within the full length A2M | |
|---|---|
| sequence of SEQ ID NO: 9 | Amino acid |
| 1,081 | R (Arg) |
| 1,082 | S (Ser) |
| 1,083 | S (Ser) |
| 1,084 | G (Gly) |
| 1,085 | S (Ser) |
| 1,086 | L (Leu) |
| 1,087 | L (Leu) |
| 1,088 | N (Asn) |
| 1,089 | N (Asn) |
| 1,090 | A (Ala) |
| 1,091 | I (Ile) |
| 1,092 | K (Lys) |
| 1,093 | G (Gly) |
| 1,094 | G (Gly) |
| 1,095 | V (Val) |
| 1,096 | E (Glu) |
| 1,097 | D (Asp) |
| 1,098 | E (Glu) |
| 1,099 | V (Val) |
| 1,100 | T (Thr) |
| 1,101 | L (Leu) |
| 1,102 | S (Ser) |
| 1,103 | A (Ala) |
| 1,104 | Y (Tyr) |
| 1,105 | I (Ile) |
| 1,106 | T (Thr) |
| 1,107 | I (Ile) |
| 1,108 | A (Ala) |
| 1,109 | L (Leu) |
| 1,110 | L (Leu) |
| 1,111 | E (Glu) |
| 1,112 | I (Ile) |
| 1,113 | P (Pro) |
| 1,114 | L (Leu) |
| 1,115 | T (Thr) |
| 1,116 | V (Val) |
| 1,117 | T (Thr) |
| 1,118 | H (His) |
| 1,119 | P (Pro) |
| 1,120 | V (Val) |
| 1,121 | V (Val) |
| 1,122 | R (Arg) |
| 1,123 | N (Asn) |

When said full length A2M sequence is the sequence of SEQ ID NO: 9, said last C-terminal amino acid of said A2M fragment is one of the following amino acids:

TABLE 5

| Position within the full length A2M | |
|---|---|
| sequence of SEQ ID NO: 9 | Amino acid |
| 1,455 | T (Thr) |
| 1,456 | D (Asp) |
| 1,457 | E (Glu) |
| 1,458 | F (Phe) |
| 1,459 | A (Ala) |
| 1,460 | I (Ile) |
| 1,461 | A (Ala) |
| 1,462 | E (Glu) |
| 1,463 | Y (Tyr) |
| 1,464 | N (Asn) |
| 1,465 | A (Ala) |
| 1,466 | P (Pro) |
| 1,467 | C (Cys) |
| 1,468 | S (Ser) |
| 1,469 | K (Lys) |
| 1,470 | D (Asp) |
| 1,471 | L (Leu) |
| 1,472 | G (Gly) |
| 1,473 | N (Asn) |
| 1,474 | A (Ala) |

More particularly:
the first N-terminal amino acid of said A2M fragment can be an amino acid, which, in the full length sequence of said A2M, is:
   at a position selected from positions 1,081-1,123,
   still more particularly from positions 1,082-1,123,
   even still more particularly from positions 1,083-1,123,
   even still more particularly from positions 1,084-1,123,
   even still more particularly from positions 1,085-1,123,
   even still more particularly from positions 1,098-1,123,
   even still more particularly from positions 1,081-1,098,
   even still more particularly from positions 1,082-1,098,
   even still more particularly from positions 1,083-1,098,
   even still more particularly from positions 1,084-1,098,
   even still more particularly from positions 1,085-1,098,

and/or, preferably and,
the last C-terminal amino acid of said A2M fragment is an amino acid, which, in the full length sequence of said A2M, is:
   one of the last twenty C-terminal amino acids (i.e., if the full length A2M sequence consists of 1,474 amino acids: one of amino acids 1,455-1,474),
   more particularly one of the last fifteen C-terminal amino acids (i.e., if the full length A2M sequence consists of 1,474 amino acids: one of amino acids 1,460-1,474),
   still more particularly one of the last ten C-terminal amino acids (i.e., if the full length A2M sequence consists of 1,474 amino acids: one of amino acids 1,465-1,474),
   still more particularly one of the last eight amino acids (i.e., if the full length A2M sequence consists of 1,474 amino acids: one of amino acids 1,467-1,474),
   still more particularly one of the last five amino acids (i.e., if the full length A2M sequence consists of 1,474 amino acids: one of amino acids 1,470-1,474),
   still more particularly one of the last four amino acids (i.e., if the full length A2M sequence consists of 1,474 amino acids: one of amino acids 1,471-1,474),
   even still more particularly the last C-terminal amino acid of said full length A2M sequence (i.e., the amino acid at position 1,474 if the full length A2M sequence consists of 1,474 amino acids).

For example, the first N-terminal amino acid of said A2M fragment is an amino acid, which, in the full length sequence of said A2M, is at position 1,098 or 1,085 or 1,084 or 1,083 or 1,123. Advantageously, the first N-terminal amino acid of said A2M fragment is an amino acid, which, in the full length sequence of said A2M, is at position 1,098 or 1,085 or 1,084 or 1,083. Very advantageously, the first N-terminal amino acid of said A2M fragment is an amino acid, which, in the full length sequence of said A2M, is at position 1,098 or 1,085.

Advantageously, the last C-terminal amino acid of said A2M fragment is the last C-terminal amino acid of said full length A2M sequence.

Please see Tables 4 and 5 above for the identity of the amino acids at the corresponding positions within the full length A2M sequence of SEQ ID NO: 9.

The application explicitly encompasses each and every combination of:
a position feature of said first N-terminal amino acid, and of
a position feature of said last C-terminal amino acid

that can be made with the above-mentioned features.

According to an alternative and/or complementary aspect of the application, the amino acid sequence of said A2M fragment consists of 330 to 400 amino acids, more particularly of 350 to 400 amino acids, still more particularly of 334 to 392 amino acids, still more particularly of 353 to 392 amino acids, still more particularly of 358 to 392 amino acids, still more particularly of 353 to 391 amino acids, still more particularly of 353 to 390 amino acids, more particularly of 353 to 377 amino acids, even still more particularly of 377, 390, 391, 392 or 353 amino acids.

According to an alternative and/or complementary aspect of the application, said A2M fragment is a C-terminal fragment of said human A2M, which does not comprise the thioester cleavage site of said A2M (in said A2M sequence of SEQ ID NO: 9, the thioester cleavage site consists of the amino acids 972-975; cf. FIGS. 4B and 4C). In other words, when considering the full length A2M sequence in a N-term to C-term orientation, the sequence of said A2M fragment is located in a region of said A2M sequence that is after the thioester cleavage site, and that does not include said thioester cleavage site.

According to an alternative and/or complementary aspect of the application, said A2M fragment has an isoelectric point (pI) of 5.90 to 7.50, more particularly of 6.00 to 7.00, still more particularly of 6.02 to 6.18.

Illustrative and advantageous A2M fragments of the application comprise the polypeptides, the amino acid sequence of which is:
the sequence extending from position 1,098 to 1,474 of a full length human A2M sequence, e.g., the sequence extending from position 1,098 to position 1,474 of sequence NM_{—}000014 (SEQ ID NO: 9), i.e., SEQ ID NO: 12;
the sequence extending from position 1,085 to 1,474 of a full length human A2M sequence, e.g., the sequence extending from position 1,085 to position 1,474 of sequence NM_{—}000014 (SEQ ID NO: 9), i.e., SEQ ID NO: 13;
the sequence extending from position 1,084 to 1,474 of a full length human A2M sequence, e.g., the sequence extending from position 1,084 to position 1,474 of sequence NM_{—}000014 (SEQ ID NO: 9), i.e., SEQ ID NO: 14;
the sequence extending from position 1,083 to 1,474 of a full length human A2M sequence, e.g., the sequence extending from position 1,083 to position 1,474 of sequence NM_{—}000014 (SEQ ID NO: 9), i.e., SEQ ID NO: 15.

The A2M polypeptides of the application can be recombinantly produced, or they can be purified and/or isolated from a native source, e.g., from the blood, plasma or serum of a hepatitis patient, more particularly a patient whose fibrosis score is of at least F2, still more particularly a patient whose fibrosis score is F2. The A2M polypeptides of the application are highly useful tools in the diagnosis, prognosis and treatment of hepatitis.

A2M Peptides of the Application:

The application provides peptides, which are sub-fragments of the A2M polypeptides of the application.

These peptides are termed the A2M peptides (or A2M sub-fragments) of the application. The A2M peptides of the application are N-terminal sub-fragments of the A2M polypeptides of the invention. Said N-terminal sub-fragment starts at the first N-terminal amino acid of said A2M polypeptide of the application, and extends (in N-term to C-term direction) for at most the next 49 contiguous amino acids.

In other words, an A2M peptide of the application is a peptide, the sequence of which is the sequence of a fragment from an A2M polypeptide of the application:
wherein the first N-terminal amino acid of said fragment is the first N-terminal amino acid of said A2M polypeptide, and
wherein said fragment consists of at most 50 amino acids.

Illustrative A2M peptides of the invention comprise peptides, the sequences of which are the sequences of:
(N-terminal) fragments of the A2M polypeptide of SEQ ID NO: 12 (the sequence of the A2M polypeptide of SEQ ID NO: 12 is the sequence of positions 1,098-1,474 of the full length human A2M of SEQ ID NO: 9);
(N-terminal) fragments of the A2M polypeptide of SEQ ID NO: 13 (the sequence of the A2M polypeptide of SEQ ID NO: 13 is the sequence of positions 1,085-1,474 of the full length human A2M of SEQ ID NO: 9);
(N-terminal) fragments of the A2M polypeptide of SEQ ID NO: 14 (the sequence of the A2M polypeptide of SEQ ID NO: 14 is the sequence of positions 1,084-1,474 of the full length human A2M of SEQ ID NO: 9);
(N-terminal) fragments of the A2M polypeptide of SEQ ID NO: 15 (the sequence of the A2M polypeptide of SEQ ID NO: 15 is the sequence of positions 1,083-1,474 of the full length human A2M of SEQ ID NO: 9);
wherein the first N-terminal amino acid of said fragments is the first N-terminal amino acid of said A2M polypeptide, and
wherein said fragment consists of at most 50 amino acids.

In other words, illustrative A2M peptides of the application comprise peptides, the sequences of which are the sequences of:
fragments of the full length human A2M of SEQ ID NO: 9, which start at position 1,098 of said full length human A2M sequence and extend (in N-term to C-direction) for at most the next 49 contiguous amino acids;
fragments of the full length human A2M of SEQ ID NO: 9, which start at position 1,085 of said full length human A2M sequence and extend (in N-term to C-direction) for at most the next 49 contiguous amino acids;
fragments of the full length human A2M of SEQ ID NO: 9, which start at position 1,084 of said full length human A2M sequence and extend (in N-term to C-direction) for at most the next 49 contiguous amino acids;
fragments of the full length human A2M of SEQ ID NO: 9, which start at position 1,083 of said full length human A2M sequence and extend (in N-term to C-direction) for at most the next 49 contiguous amino acids.

According to an embodiment of the application, said fragment of at most 50 amino acids consists of at most 45 amino acids or at most 40 amino acids, or at most 35 amino acids, or at most 30 amino acids, or at most 25 amino acids, or of at most 20 amino acids, or at most 19 amino acids, or at most 18 amino acids, or at most 17 amino acids, or at most 16 amino acids, or at most 15 amino acids, or at most 14 amino acids, or at most 13 amino acids, or at most 12 amino acids, or at most 11 amino acids, or at most 10 amino acids, or at most 9 amino acids, or at most 8 amino acids, or at most 7 amino acids, or at most 6 amino acids, or at most 5 amino acids.

More particularly, said fragment of at most 50 amino acids may consist of at most 20 amino acids, or at most 19 amino acids, or at most 18 amino acids, or at most 17 amino acids, or at most 16 amino acids, or at most 15 amino acids, or at most 14 amino acids, or at most 13 amino acids, or at most 12 amino acids, or at most 11 amino acids, or at most 10 amino acids, or at most 9 amino acids, or at most 8 amino acids, or at most 7 amino acids.

Still more particularly, said fragment of at most 50 amino acids may consist of at most 16 amino acids, or at most 15 amino acids, or at most 14 amino acids, or at most 13 amino acids, or at most 12 amino acids, or at most 11 amino acids, or at most 10 amino acids, or at most 9 amino acids, or at most 8 amino acids, or at most 7 amino acids.

Illustrative A2M peptides of the invention notably comprise:
the peptide of SEQ ID NO: 1 (EVTLSAYITI), which is the N-terminal 10aa fragment from the A2M polypeptide of SEQ ID NO: 12 (i.e., positions 1,098-1,107 within the full length human A2M sequence of SEQ ID NO: 9);
the peptide of SEQ ID NO: 2 (SLLNNAIK), which is the N-terminal 8aa fragment from the A2M polypeptide of SEQ ID NO: 13 (i.e., positions 1,085-1,092 within the full length human A2M sequence of SEQ ID NO: 9);
the peptide of SEQ ID NO: 3 (SLLNNAIKGGVEDE), which is the N-terminal 14aa fragment from the A2M polypeptide of SEQ ID NO: 13 (i.e., positions 1,085-1,098 within the full length human A2M sequence of SEQ ID NO: 9);
the peptide of SEQ ID NO: 4 (SLLNNAIKGG), which is the N-terminal 10aa fragment from the A2M polypeptide of SEQ ID NO: 13 (i.e., positions 1,085-1,094 within the full length human A2M sequence of SEQ ID NO: 9);
the peptide of SEQ ID NO: 5 (GSLLNNAIK), which is the N-terminal 9aa fragment from the A2M polypeptide of SEQ ID NO: 13 (i.e., positions 1,084-1,092 within the full length human A2M sequence of SEQ ID NO: 9);
the peptide of SEQ ID NO: 6 (GSLLNNAIKGGVEDE), which is the N-terminal 15aa fragment from the A2M polypeptide of SEQ ID NO: 14 (i.e., positions 1,084-1,098 within the full length human A2M sequence of SEQ ID NO: 9);
the peptide of SEQ ID NO: 7 (SGSLLNNAIK), which is the N-terminal 10aa fragment from the A2M polypeptide of SEQ ID NO: 15 (i.e., positions 1,083-1,092 within the full length human A2M sequence of SEQ ID NO: 9);
the peptide of SEQ ID NO: 8 (SGSLLNNAIKGGVEDE), which is the N-terminal 16aa fragment from the A2M polypeptide of SEQ ID NO: 15 (i.e., positions 1,083-1,098 within the full length human A2M sequence of SEQ ID NO: 9);
the peptide of SEQ ID NO: 17 (EVTLSAY), which is the N-terminal 7aa fragment from the A2M polypeptide of SEQ ID NO: 12 (i.e., positions 1,098-1,104 within the full length human A2M sequence of SEQ ID NO: 9).

The A2M peptides of the application can be produced by peptide synthesis (e.g., by solid-phase synthesis) or by recombinant expression. They are notably useful as antigens for the production of antibodies and antibody fragments, which specifically bind to an A2M polypeptide of the application. For such an application, an A2M peptide of the application may advantageously be linked to (e.g., by covalent linkage) or conjugated to, or bound to at least one immunogenicity carrier, i.e., to at least one immunogenic molecule, which contains antigenic determinants recognized by T cells. Immunogenicity carriers may render immunogenic a polypeptide or peptide that is linked or conjugated to it, or may enhance the immunogencity of said polypeptide or peptide. Illustrative immunogenicity carriers comprise lipids, proteins, polypeptides and peptides, such as Keyhole Limpet Hemocyanin (KLH), Horseshoe Crab Hemocyanin (HCH), and Bovine Serum Albumin (BSA). Preferably, said immunogenicity carrier(s) is(are) linked to the C-terminal extremity of said A2M peptide of the application, whereby the N-terminal amino group of said peptide remains free.

The application also encompasses any composition, which comprises:
at least one A2M peptide of the application (optionally linked to (e.g., by covalent linkage) or conjugated to or bound to at least one immunogenicity carrier, more particularly to at least one protein, polypeptide or peptide, which is an immunogenicity carrier, such as KLH or BSA, more particularly KLH); and
at least one immunologic adjuvant, such as Freund's complete adjuvant, Freund's incomplete adjuvant, TITERMAX® gold adjuvant, alum, bacterial lipopolysaccharide (LPS).

The A2M peptides of the application are also useful as affinity ligands for screening for such antibodies or antibody fragments. For such an application, an A2M peptide of the application may advantagously be linked to a protein, polypeptide or peptide, which can bind solid support (such as a glass support or a plastic support e.g., a polystyrene solid support), for example to Bovine Serum Albumin (BSA).

A2M Compounds of the Application (A2M Polypeptides of the Application or A2M Peptides of the Application, Linked to Other Structural Components):

The application also encompasses any compound, which consists of:
at least one A2M polypeptide of the application, as above-described, or at least one A2M peptide of the application, as above-described,

linked to (e.g., by covalent linkage), or otherwise conjugated, bound or coupled to
one or several other structural components (i.e., one or several structural components other than A2M fragments of the application),

provided that said A2M compound:
is not human A2M, and
is not a human A2M fragment, which would comprise an A2M polypeptide of the application and be a bigger A2M fragment than said A2M polypeptide of the application.

Such a compound is herein termed an A2M compound of the application.

Said other structural components are molecules, atoms, ions or complexes. A structural component can e.g., be a polypeptide, but in such a case, this polypeptide is not an A2M fragment of the application. For example, an A2M compound of the application can comprise:
at least one A2M fragment of the application (as above-described), and
another A2M fragment, which is not an A2M fragment of the application but which, in the full length A2M sequence, is not a fragment that is immediately contiguous to said A2M fragment of the application.

Nevertheless, a particular embodiment of the application is that none of said other structural components are fragments of human A2M.

Such other structural components are for example structural components that are useful:
for polypeptide detection,
for the recombinant production of a polypeptide,
for the use of a polypeptide as a screening tool, more particularly as an antibody screening tool,
for the use of a polypeptide as an antigen,

more particularly at least one structural component that is useful for the recombinant production of a polypeptide, e.g., for purification and/or isolation of a recombinantly produced polypeptide.

For example, said one or several other structural components can be:
at least one label that can be useful for polypeptide detection (e.g., biotin, a radioactive element, a fluorophore), and/or
at least one linker (such as an antibody and antibody fragment) for binding or linking an A2M polypeptide or peptide of the application onto a solid support, such a glass support or a plastic support (e.g., a plaque, a microplaque or a chip), and/or
at least one tag for purification or isolation of recombinantly produced polypeptide or peptide (e.g., at least one peptide tag such as at least one His tag), and/or
at least one protein, polypeptide or peptide, which is useful for ELISAs, immunoblot or immunocytochemistry, more particularly at least one protein, polypeptide or peptide, which can bind a solid support such as a glass support or a plastic support (e.g., a polystyrene solid support), for example Bovine Serum Albumin (BSA), and/or
at least one immunogenicity carrier, i.e., to at least one immunogenic molecule (e.g., a lipid, protein, polypeptide or peptide), which contains antigenic determinants recognized by T cells, and which may thereby render a polypeptide or peptide immunogenic, or enhance its immunogenicity, such as Keyhole Limpet Hemocyanin (KLH), Horseshoe Crab Hemocyanin (HCH), serum albumin, Bovine Serum Albumin (BSA), bovine thyroglobulin, soybean trypsin inhibitor [e.g., by using a bifunctional or derivatizing agent, for example, maleimidobenzoyl sulfosuccinimide ester (conjugation through cysteine residues), N-hydroxysuccinimide (through lysine residues), glutaraldehyde, succinic anhydrid or SOCl₂].

Hence, said other structural components can e.g., be selected from the group consisting of:
peptide tags for purification or isolation of recombinantly produced polypeptides (such as at least one His tag),
antibodies, antibody fragments,
biotin,
fluorophores,
radioactive elements,
at least one protein, polypeptide or peptide, which can bind a solid support such as a glass support or a plastic support (e.g., a polystyrene solid support), such as BSA,
proteins, polypeptides or peptides, which are immunogenicity carriers, such as KLH, HCH, BSA;

more particularly
peptide tags for purification or isolation of recombinantly produced polypeptides (such as at least one His tag),
radioactive elements,
at least one protein, polypeptide or peptide, which can bind a solid support such as a glass support or a plastic support (e.g., a polystyrene solid support), such as BSA,
proteins, polypeptides or peptides, which are immunogenicity carriers, such as KLH, HCH, serum albumin, BSA;

still more particularly peptide tags for purification or isolation of recombinantly produced polypeptide (such as at least one His tag), serum albumin, BSA and KLH.

According to an embodiment of the application, said A2M compound consists of:
at least one A2M polypeptide of the application, as above-described,

linked to (e.g., by covalent linkage), or otherwise bound or coupled to
one or several other structural components, which comprise or consist of peptide tags for purification or isolation of recombinantly produced polypeptide (such as His tags),

provided that said A2M compound:
is not human A2M, and
is not a human A2M fragment, which would comprise an A2M polypeptide of the application and be a bigger A2M fragment than said A2M polypeptide of the application.

According to an embodiment of the application, said A2M compound consists of:
at least one A2M peptide of the application, as above-described,

linked to (e.g., by covalent linkage), or otherwise conjugated, bound or coupled to
one or several other structural components, which comprise or consist of:
   proteins, polypeptides or peptides, which can bind a solid support such as a glass support or a plastic support (e.g., a polystyrene solid support), such as BSA,
   proteins, polypeptides, peptides, which are immunogenicity carriers, such as KLH, HCH, serum albumin, BSA, more particularly KLH or BSA, still more particularly KLH;

provided that said A2M compound:
is not human A2M, and
is not a human A2M fragment, which would comprise an A2M polypeptide of the application and be a bigger A2M fragment than said A2M polypeptide of the application.

According to an embodiment of the application, said other structural components are linked to (e.g., by covalent linkage), or otherwise conjugated, bound or coupled to the C-terminal extremity of said A2M polypeptide or peptide of the application (e.g., to the last C-terminal amino acid of said C-terminal extremity).

According to a particular embodiment of the application, none of said other structural components are linked to (e.g., by covalent linkage), or otherwise conjugated, bound or coupled to the first N-terminal amino acid of the N-terminal extremity of said A2M polypeptide or peptide of the application. The amino group of said first N-terminal amino acid remains thereby free.

The application also encompasses any A2M polypeptide of the application, or A2M peptide of the application, or A2M compound of the application, which is linked or otherwise bound to a solid support, such as a glass support or a plastic support (e.g., a polystyrene solid support). Said solid support can e.g., a chip, or in the form of a plaque or microplate, e.g., a plaque or microplate comprising wells, such as an ELISA plaque or microplate.

The application also encompasses any non-human animal, e.g., any non-human mammal, e.g., any non-human primate or any rodent (e.g., any rabbit), the body of which contains at least one A2M peptide of the application and/or at least one A2M compound of the application, wherein said at least one A2M compound comprises at least one A2M peptide of the application (e.g., at least one A2M compound comprising at least one A2M peptide of the application and at least one immunogenicity carrier).

Nucleic Acids, Vectors, Host Cells of the Application:

The application also encompasses any nucleic acid, the sequence of which codes for an A2M polypeptide of the application, or for an A2M peptide of the application, or for an A2M compound of the application, in accordance with the universal genetic code and taking due account of its redundancy. When the one or several of said other structural components that are contained in the A2M compound of the application are not all made of amino acids, the nucleic acid of the application does of course code for the portion of said A2M compound, which is made of amino acids, i.e., for at least one A2M polypeptide or peptide of the application and for those of said other structural components, which comprise or are made of amino acids. In an embodiment of the application, the coding sequence of said nucleic acid consists of a sequence coding for an A2M polypeptide of the application, or for an A2M peptide of the application, or for an A2M compound of the application.

The application further encompasses vectors, more particularly nucleic acid vectors, still more particularly recombinant vectors. Said vectors are vehicles, in which a nucleic acid has been inserted. The vector itself usually is a nucleic acid molecule comprising a nucleic acid backbone into which the nucleic acid to be transferred is inserted. Said vectors are notably useful for transferring a coding nucleic acid into a cell. Vectors notably comprise transformation vectors (to transform bacterial cells), transfection vectors (to transfect eukaryotic cells), and expression vectors (to express the transferred nucleic acid). Illustrative vectors notably comprise plasmids and viral vectors (such as retroviruses). An expression vector usually comprises an expression cassette, wherein a coding nucleic acid is operably linked to control sequence(s). The terms “expression cassette”, “control sequence(s)”, “operably linked” are intended in accordance with their ordinary meaning in the field. For example, the term “expression cassette” means a nucleic acid construct, which contains a coding nucleic acid and the control sequence(s) required for expression of said coding nucleic acid. Such control(s) sequence(s) notably comprise at least one promoter. They usually further include transcriptional and translational stop signals. They may further include a leader sequence and/or a polyadenylation sequence and/or a signal peptide sequence and/or a transcription terminator. The expression “operably linked” implies that the control sequence is a cis-acting regulatory element. The person of ordinary skill in the art understands that a regulatory element is appropriately placed with respect to said coding nucleic acid such that said control sequence can direct or modulate the expression of the polypeptide encoded by said nucleic acid. For example, a promoter is placed in 5′ of the coding nucleic acid so that it can drive the expression of said coding nucleic acid.

The application more particularly encompasses any vector, more particularly any expression and/or recombinant vector:
which comprises a nucleic acid, the sequence of which codes for and/or
which comprises a coding nucleic acid inserted therein, wherein the expression product of said coding nucleic acid is and/or
which comprises a nucleic acid coding for the expression of

one or several A2M polypeptide(s) of the application, and/or one or several A2M peptide(s) of the application, and/or one or several A2M compound(s) of the application, e.g., an A2M polypeptide of the application that is linked to a tag that can be useful in the purification or isolation of a recombinantly produced polypeptide, e.g., a His tag.

A coding nucleic acid is a nucleic acid, the sequence of which codes for a polypeptide or peptide in accordance with the universal genetic code and taking due account of its redundancy.

Said coding nucleic acid does not code a full length human A2M, and does not code any of the thioester-cleaved and bait-cleaved fragments of said human A2M. Hence, the sequence of the coding nucleic acid may consist of a sequence coding for one or several A2M polypeptide(s) of the application, and/or one or several A2M peptide(s) of the application, and/or one or several A2M compound(s) of the application, e.g., for an A2M polypeptide of the application that is linked to a tag that can be useful in the purification or isolation of a recombinantly produced polypeptide, e.g., a His tag.

The A2M polypeptide(s), peptide(s) or compound(s) coded by said coding nucleic acid is(are) expressed in isolated form. Nevertheless, once said polypeptide or peptide or compound is expressed by said vector, it may bind or link to other structural elements, such as e.g., structural components and/or organelles of the host cell, into which said vector has been transferred.

The application more particularly encompasses any expression vector, which, when transferred into a host cell (such as E. coli or S. cerevisae), expresses an A2M polypeptide of the application (in isolated form), or an A2M peptide of the application (in isolated form), or an A2M compound of the application (in isolated form), e.g., an A2M polypeptide of the application that is linked to a tag that can be useful in the purification or isolation of a recombinantly produced polypeptide, e.g., a His tag.

The expression “in isolated form” means that the sequence of the coding nucleic acid that is inserted into said vector consists of a sequence coding for an A2M polypeptide of the invention, or for an A2M peptide of the application, or for an A2M compound of the application. Nevertheless, once said polypeptide or peptide or compound is expressed by said vector in a host cell, it may of course link or bind to other structural elements, such as e.g., structural components and/or organelles of the host cell.

The application further encompasses any host cell:
which comprises or expresses an A2M polypeptide of the application and/or an A2M peptide of the application and/or an A2M compound of the application, and/or
which comprises a nucleic acid of the application and/or a vector of the application.

Such host cells may notably be produced by genetic engineering. The host cell may be a prokaryote or a eukaryote. Illustrative prokaryotes notably comprise bacterial cells, such as E. coli. Illustrative eukaryotes comprise a mammalian cell, a non-human mammalian cell, a non-human animal cell, a plant cell, a yeast cell (such as S. cerevisiae). Means for genetically engineering a host cell are known in the art. They for example include the electroporation of the nucleic acid to be incorporated into said cell or the growth of host cells in the presence of vector(s) comprising said nucleic acid.

The nucleic acids, vectors and host cells of the application are notably useful for producing an A2M polypeptide, or an A2M peptide, or an A2M compound of the application, e.g., for in vitro producing and/or for recombinantly producing an A2M polypeptide, or an A2M peptide, or an A2M compound of the application.

The application therefore encompasses a method of production of an A2M polypeptide or an A2M peptide or an A2M compound of the application, which comprises:
growing a genetically engineered host cell of the application in a cell culture, and
recovering said A2M polypeptide or A2M peptide or A2M compound from the supernatant of said culture and/or from said grown cells.

The application also encompasses primers and probes. The notion of primer is known to the person of ordinary skill in the art. For example, it includes any oligonucleotide able to anneal to a target nucleic acid under suitable stringency conditions (such as conditions of high stringency), and allowing for polym erase strand elongation. The typical length of said primer is 15-30 nucleotides (nt), preferably 18, 19, 20, 21, 22, 23, 24 or 25 nt. The nucleotide sequence of the primer may e.g., be at least 85%, more particularly at least 90%, still more particularly at least 95%, still more particularly 100% identical to:
the sequence of the same length that is the 5′ end extremity of said target nucleic acid (forward primer), or
the sequence that is the complementary sequence of the sequence of the same length that is the 3′ end extremity of said target nucleic acid (reverse primer),

whereby the sequence of the amplified nucleic acid by said primer pair is a sequence consisting of the sequence of a nucleic acid of the application. The notion of probe is also known to the person of ordinary skill in the art. For example, it includes any oligonucleotide able to anneal to a target nucleic acid under the desired hybridization conditions (such as conditions of high stringency). The typical length of said probe is 20-55 nucleotides (nt), preferably 15-60 nt, more preferably 20-55 nt, more preferably 30-50 nt, more preferably 35-45 nt. Preferably, said probe is fluorescently labelled and/or is in a TAQMAN™ probe format (hydrolysis probes) and/or in a molecular BEACON™ format (beacon probes or molecular beacon probes) and/or in a SCORPION™ probe format. The portion of the probe that anneals to said target nucleic acid may e.g., be a nucleotide sequence that is at least 85%, more particularly at least 90%, still more particularly at least 95%, still more particularly 100% identical to a sequence of the same length that is contained in said target nucleic acid or to the complementary sequence thereof. Sequence identity percentages are to be understood in accordance with their ordinary meaning in the field (schematically: the percentage of identical matches between the two sequences over the optimum alignment of the two sequences; see e.g., Needleman and Wunsch 1970). In the application, said target nucleic acid is a nucleic acid coding for an A2M polypeptide of the application, such as a nucleic acid coding for the polypeptide of SEQ ID NO: 12, 13, 14 or 15. According to an embodiment of the application, the primers and/or the probes specifically hybridize to a nucleic acid of the application (or to the complementary sequence thereof) under conditions of high stringency. An example of conditions of high stringency is hybridization to filter-bound DNA in 5× Saline-Sodium Citrate (SSC), 2% Sodium Dodecyl Sulfate (SDS), 100 micrograms/mL single stranded DNA at 55-65° C. for 8 hours, and washing in 0.2×SSC and 0.2% SDS at 60-65° C. for thirty minutes. The application encompasses a set of oligonucleotides, comprising at least one primer pair of the application, i.e., a forward primer and a reverse primer of the application. Said set may further comprise at least one probe of the invention. The primers and/or probes of the application are notably useful for amplifying, e.g., by PCR, a nucleic acid of the invention, e.g., from a biological sample (e.g., from blood, plasma or serum) of a hepatitis patient.

Binding Reactants of the Application (Antibodies and Antibody Fragments):

The application also relates to any antibody or antibody fragment, which specifically binds to an A2M polypeptide or peptide of the application.

According to a particular embodiment, the antibody or antibody fragment of the application binds to said A2M polypeptide or peptide of the application, without binding to the full length human A2M protein. More particularly, the antibody or antibody fragment of the application binds to the N-terminal extremity of said A2M polypeptide or peptide of the application, without binding to the full length human A2M protein. Still more particularly, the antibody or antibody fragment of the application binds to the N-terminal extremity of said A2M polypeptide or peptide of the application at a site or epitope, which comprises the N-terminal amino group of said A2M polypeptide or peptide. By convention, when the person of ordinary skill in the art is drawing an amino acid chain:
the amino acid bearing the free amino group (i.e., the free alpha-amino group) is drawn to the left-hand side and is the first amino acid (first N-terminal amino acid), and
the amino acid bearing the free carboxyl group (i.e., a free alpha-carboxyl group) is drawn to the right-hand side and is the last amino acid (last C-terminal amino acid).

The N-terminal amino group of said A2M polypeptide or peptide is the amino group, which the person of ordinary skill in the art considers to be the free amino group of the first N-terminal amino acid of said A2M polypeptide or peptide, when drawing the amino acid chain of said A2M polypeptide or peptide in an N-term to C-term orientation, i.e., the amino group which the person of ordinary skill in the art considers to be the N-terminal alpha-amino group. The antibody or antibody fragment of the application specifically binds to said A2M polypeptide or peptide of the application, when the N-terminal amino group of said A2M polypeptide or peptide is free. The term “free” is intended in accordance with its ordinary meaning in the field. A free N-terminal amino group is not engaged in a peptide bond (i.e., it is not forming a peptide bond). More generally, a free N-terminal amino group is not engaged in any covalent bond. Still more generally, a free N-terminal amino group is not engaged in any chemical bond, at least not in a chemical bond, which may prevent an antibody from binding thereto. In other words, a free N-terminal bond is unbound such as to be available for antibody binding thereto. When said N-terminal amino group is not free, e.g., when it is forming a peptide bound with another amino acid, the antibody or antibody fragment of the application does not bind to the amino acid chain of the A2M polypeptide or peptide of the application. For example, when the amino acid chain of said A2M polypeptide or peptide of the application is inserted within another amino acid sequence, e.g., when it is contained within the full length sequence of human A2M, the N-terminal amino group of said A2M polypeptide or peptide sequence is not free: it forms a peptide bond with the amino acid, which immediately precedes it within the full length human A2M sequence. The antibody or antibody fragment of the application does not bind to such an inserted A2M polypeptide or peptide amino acid chain. For the same reason:
the antibody or antibody fragment of the application does not bind to the thioester-cleaved fragments of the human A2M protein, more particularly to the C-terminal thioester-cleaved fragment;
the antibody or antibody fragment of the application does not bind to the bait-cleaved fragments of the human A2M protein, more particularly to the C-terminal bait-cleaved fragment.

More particularly, the application encompasses any antibody or antibody fragment, which binds to the A2M polypeptide of SEQ ID NO: 12, 13, 14 or 15, without binding to the full length human A2M protein of SEQ ID NO: 9.

An antibody of the application can be a polyclonal antibody or a monoclonal antibody, more particularly a monoclonal antibody.

An antibody fragment of the application can be any antibody fragment, which has retained the binding properties of an antibody of the application, more particularly its binding specificity. Illustrative antibody fragments of the application comprise Fab fragments, F(ab′)2, Fv, Fab/c (consisting of one Fab and a complete Fc). Antibody fragments of the application also encompass constructs made of antibody fragments, such as a single chain Fv (scFv), i.e., a construct wherein the H-chain V-region is linked to the L-chain V-region, e.g., through an an appropriate linker.

The application encompasses any hybridoma, which produces (or secretes) a monoclonal antibody of the application, as well as any composition comprising such an hybridoma.

The application further encompasses a method of producing an antibody of the application, which comprises:
immunizing an animal, more particularly a non-human animal, with at least one A2M polypeptide of the application, or with at least one A2M peptide of the application or at least one A2M compound of the application or with an antigenic functional derivative thereof, more particularly with at least one A2M peptide of the application or with at least one A2M compound of the application that comprises at least one A2M peptide of the application,
recovering the antibodies from said immunized animal.

To immunize said animal, the person may use any A2M polypeptide, peptide or compound of the application, which he/she finds appropriate. Advantageously, the person of ordinary skill in the art may use one or several of the A2M peptides of the application, such as one or several of the peptides of SEQ ID NOs: 1-8. The A2M peptide(s) may be linked, preferably at its/their C-terminal end, to an immunogenicity carrier, such as KLH, serum albumin, BSA, bovine thyroglobulin, soybean trypsin inhibitor, e.g., by using a bifunctional or derivatizing agent, for example, maleimidobenzoyl sulfosuccinimide ester (conjugation through cysteine residues), N-hydroxysuccinimide (through lysine residues), glutaraldehyde, succinic anhydrid or SOCl₂.

Means to produce antibodies are known to the person or ordinary skill in the art. Animals can be immunized with an A2M polypeptide of the application or with an A2M peptide of the application or with an A2M compound of the application or with an antigenic functional derivative thereof, more particularly with an A2M peptide of the application or with an A2M compound of the application that comprises at least one A2M peptide of the application. Appropriate animals notably comprise mammals, more particularly non-human mammals, such as rabbits. For example, the animal is injected intraperitoneally or subcutaneously with said A2M polypeptide, peptide or compound or an antigenic functional derivative thereof. Said A2M polypeptide, peptide or compound or antigenic functional derivative thereof, may be diluted with, or suspended in an appropriate volume of PBS (Phosphate-Buffered Saline), physiological saline or the like. An appropriate volume of a standard adjuvant can be mixed with the product, if necessary or desired. Illustrative standard adjuvants notably comprise Freund's (complete or incomplete) adjuvant, mineral gels such as aluminium hydroxide, surface active substances such as lysolecithin, pluronic polyols, polyanions, peptides, oil emulsions, KLH, dinitrophenol, and potentially useful human adjuvants such as BCG (bacille Calmette-Guerin) and Corynebacterium parvum. Emulsification can be performed and the solution is administered to mammals, e.g., several times every 4 to 21 days. Polyclonal antibodies are heterogeneous populations of antibody molecules, which can be derived from the sera of immunized animals. Monoclonal antibodies, which are homogeneous populations of antibodies to a particular antigen, may be obtained by any technique which provides for the production of antibody molecules by continuous cell lines in culture. These include, but are not limited to the hybridoma technique of Köhler and Milstein (Köhler and Milstein 1975; U.S. Pat. No. 4,376,110), the human B-cell hybridoma technique (Kosbor et al. 1983; Cole et al. 1983) and the EBV-hybridoma technique (Cole et al. 1985). Such antibodies may be of any immunoglobulin class including IgG, IgM, IgE, IgA, IgD and any subclass thereof. The hybridoma producing a monoclonal antibody of the application may be cultivated in vitro or in vivo.

The method of producing an antibody the application may further comprise the step of purifying or isolating those antibodies, which specifically binds to said A2M polypeptide, peptide or compound of the application. More particularly, the method of the application may comprise the step of purifying or isolating those antibodies:
which specifically binds to said A2M polypeptide, peptide or compound of the application, when the N-terminal amino group of said A2M polypeptide, peptide or compound is free, and
which does not bind to the amino acid chain of said A2M polypeptide, peptide or compound, when the N-terminal amino group of said A2M polypeptide, peptide or compound is not free.

For example, the method of the application may comprise the step of purifying or isolating those antibodies:
which specifically binds to said A2M polypeptide, peptide or compound of the application, when the N-terminal amino group of said A2M polypeptide, peptide or compound is free, and
which does not bind to the full length human A2M protein (e.g., the A2M protein of SEQ ID NO: 9 or 10) and/or to the C-terminal thioester-cleaved fragment thereof and/or to the C-terminal bait-cleaved fragment thereof.

Isolation and/or purification of the antibodies can be performed using affinity column(s), for example a protein A column (e.g., for purification and/or isolation of polyclonal antibodies) or a protein B column (e.g., for purification and/or isolation of a monoclonal antibody). Other standard methods for isolation and/or purification of antibodies may be used. For example, a chromatography column other than the above affinity column, a filter, ultrafiltration, a method of salting out, dialyses and the like may be appropriately selected and combined for use, so that antibodies can be isolated and purified (Harlow and Lane 1988 “Antibodies A Laboratory Manual”).

The application also encompasses a kit comprising at least one antibody or antibody fragment of the application. Such a kit may further comprise one or several of the following means:
means suitable for collecting or obtaining a biological sample from a human patient, e.g., means for the collection of blood, and/or means for separating plasma or serum from blood, such as syringe(s) and/or tube(s) (e.g., centrifugation tube(s)), and/or
means for purifying said biological sample, more particularly means for depleting albumin and/or immunoglobulin G from said biological sample (e.g., from blood, plasma or serum), such as a resin (e.g., agarose beads) allowing a selective binding of albumin and/or immunoglobulin G from a biological sample, e.g., cross-linked agarose beads with covalently attached CIBACRON BLUE F3GA™ dye and protein A (such cross-linked agarose beads are e.g., available in the AURUM® kit, Bio-Rad, Hercules, Calif.), and/or
means suitable for denaturing proteins, e.g., heating means or a surfactant that denatures proteins, such as Sodium Dodecyl Sulfate (SDS), TRITON®, urea, thiourea.

Said kit may further comprise any means that are useful for the performance of antibody-antigen reactions and/or for their detection.

Illustrative Applications of the Means of the Application:

The means of the invention are notably useful in the field of hepatitis, more particularly of viral hepatitis, such as Hepatitis C Virus (HCV) infection and/or Hepatitis B Virus (HBV) infection and/or Hepatitis D Virus (HDV) infection, more particularly of HCV infection.

The means of the invention are more particularly useful for determining the extent of liver fibrosis, i.e., for staging fibrosis. The extent of liver fibrosis is staged according to a scoring system, known as the Metavir score (F score), as follows:
no fibrosis (Metavir F score=F0);
mild fibrosis (Metavir F score=F1);
moderate fibrosis (Metavir F score=F2);
advanced fibrosis (Metavir F score=F3);
severe fibrosis or cirrhosis (Metavir F score=F4).

Determining the fibrosis stage is of upmost clinical importance. Indeed, due to the highly noxious secondary effects of the current treatments, the fibrosis stage is the main element on which is based the decision to administer a treatment. The F2 Metavir score is generally chosen as a threshold for treatment of HCV infection. The fibrosis score thereby is a prognostic tool. Determining the fibrosis stage of a patient before and/or during and/or after a treatment also enables to determine whether this treatment is useful in reducing the extent of fibrosis. Various markers can be used either individually on their own, or in combination with other marker(s) and/or with clinical feature(s) [such as age, sex, etc.], to determine the fibrosis score from a non-invasive biological sample, such as blood, serum, plasma. Human A2M is one of such markers. Please see the background section above. Please also see the French patent application FR 1151022 filed on 9 Feb. 2011, and its foreign counter-parts, notably its US counter-part (U.S. 61/440,986 filed on 9 Feb. 2011), the respective contents of which being herein incorporated by reference. Said French patent application FR 1151022 or foreign counterpart thereof describe a combination of markers, which enables to score the fibrosis stage, more particularly to determine whether the fibrosis score of a hepatic patient is of at most F1 or of at least F2. This combination is the combination of the level of transcription or translation of the following genes:
SPP1, and
at least one gene among A2M and VIM, and
at least one gene among IL8, CXCL10 and ENG, and
optionally, at least one gene among the following list of sixteen genes: IL6ST, p14ARF, MMP9, ANGPT2, CXCL11, MMP2, MMP7, S100A4, TIMP1, CHI3L1, COL1A1, CXCL1, CXCL6, IHH, IRF9, MMP1,

wherein SPP1 is secreted phosphoprotein 1; VIM is vimentin; IL8 is interleukin-8; CXCL10 is C—X—C motif chemokine 10; ENG is endoglin; IL6ST is interleukin-6 signal transducer; p14ARF is cyclin-dependent kinase 2A inhibitor; MMP9 is matrix metalloproteinase 9; ANGPT2 is angiopoietin-2; CXCL11 is C—X—C motif chemokine 11; MMP2 is matrix metalloproteinase 2; MMP7 is matrix metalloproteinase 7; S100A4 is protein S100-A4; TIMP1 is metalloproteinase inhibitor 1; CHI3L1 is chitinase-3-like protein; COL1A1 is collagen alpha-1(I) chain; CXCL1 is growth-regulated alpha protein C—X—C motif chemokine 1; CXCL6 is C—X—C motif chemokine 6; IHH is Indian hedgehog protein; IRF9 is interferon regulatory factor 9; MMP1 is matrix metalloproteinase-1.

The A2M polypeptides of the application are differentially abundant depending on the stage of hepatic fibrosis. More particularly, the content or concentration of an A2M polypeptide of the invention is higher in those patients, whose fibrosis score is of at least F2, compared to those patients, whose fibrosis score is of at most F1 (Metavir scoring system). This difference is notably observed in a biological fluid from said patients (e.g., blood, serum, plasma, urine or cerebrospinal fluid (CSF), more particularly blood, serum or plasma). Please see the examples below. Therefore, the A2M polypeptides of the invention are markers, which can be used either individually or in combination with each other and/or with other marker(s) and/or clinical feature(s), such as in the above-mentioned marker combination. Detecting an A2M polypeptide of the application, and/or measuring its content or concentration, enable to, or contribute to enable:
to detect liver fibrosis and/or
to determine a fibrosis score and/or
to determine whether the fibrosis score is of at most F1 or of at least F2.

For said A2M polypeptide detection and/or measure, antibody(ies) or antibody fragment(s) of the application can be used. To produce such antibodies, A2M peptide(s) of the invention can be used.

Therefore, the application encompasses the in vitro use of at least one antibody or antibody fragment of the application or of at least one kit of the application:
for detecting at least one polypeptide or peptide of the application (or a polypeptide or peptide having the same starting position), and/or
for detecting hepatitis and/or liver fibrosis, and/or
for measuring the content of concentration at least one polypeptide of the application (or a polypeptide having the same starting position), and/or
for scoring a liver fibrosis stage, and/or
for determining whether a liver fibrosis is at a stage, which scores at most F1 or at a stage, which scores at least F2, in accordance with the Metavir scoring system.

The application also encompasses a method:
for detecting at least one polypeptide of the application (or a polypeptide having the same starting position), and/or
for detecting hepatitis and/or liver fibrosis, and/or
for measuring the content of concentration at least one polypeptide of the application (or a polypeptide having the same starting position),

which comprises:
placing at least one antibody or antibody fragment of the application in contact with a biological sample of a biological fluid of a human being under conditions enabling reactions of the antibody-antigen type, more particularly under conditions enabling the binding of said at least one antibody to its epitope,
detecting the antibody or antibody fragment thereby bound, and/or measuring the concentration of antibody or antibody fragment thereby bound.

The presence of bound antibody or antibody fragment is indicative of the fact that said human being has at least one A2M polypeptide of the application in his/her biological fluid. The presence of bound antibody or antibody fragment is also indicative of the fact that said human being has hepatitis and/or liver fibrosis. The concentration of bound antibody or antibody fragment is the concentration of the A2M polypeptide of the application, onto which said antibody or antibody fragment binds.

Said biological sample of a biological fluid of a human being can more particularly be a biological sample of blood, serum, plasma, urine or cerebrospinal fluid (CSF), still more particularly of blood, serum, plasma. Said human being preferably is a human being infected by at least one hepatitis virus, still more particularly by at least HCV, even still more particularly by HCV. Said human being may e.g., be afflicted by a chronic hepatitis. The detected presence or absence of, or the measured concentration of, bound antibody or antibody fragment can be compared to the presence/absence, or to the concentration of, bound antibody or antibody fragment, respectively, which is observed:
in a cohort of hepatitis patients, more particularly of HCV patients, who score at most F1 in accordance with the Metavir scoring system, and in
in a cohort of hepatitis patients, more particularly of HCV patients, who score at least F2 in accordance with the Metavir scoring system.

This comparison can be made by any statistical analysis tool that the person of ordinary skill in the art may find appropriate to classify said detected presence or absence, or said measured concentration, respectively, in the cohort of the cohort of patients, who are at most F1, or in the cohort of the patients, who are at least F2, thereby attributing a score of “at most F1” or “of at least F2” to said human being.

A similar methodology but performed with a range of cohorts, which corresponds to the full range of Metavir scores, enables to score said human being as being F0, F1, F2, F3, F4. Of course, partial scoring range may equally be performing (such as e.g., at most F1, F2, at least F3).

Hence, the method of the application also is a method:
for scoring a liver fibrosis stage, and/or
for determining whether a liver fibrosis is at a stage, which scores at most F1 or at a stage, which scores at least F2, in accordance with the Metavir scoring system.

Since the decision to administer an anti-hepatitis and/or anti-HCV and/or anti-fibrosis treatment is usually directly linked to the fact that the patient has a liver fibrosis that scores at least F2 according to the Metavir scoring system, the method of the application to determine whether a liver fibrosis is at a stage, which scores at most F1 or at a stage, which scores at least F2, in accordance with the Metavir scoring system, also is a method to administer an anti-hepatitis and/or anti-HCV and/or anti-fibrosis treatment in a patient in need thereof, which comprises:
determining by a means of the application (e.g., using at least one antibody or antibody fragment of the application), whether the patient's liver is at a fibrosis stage, which scores at most F1 or at a stage, which scores at least F2 (in accordance with the Metavir scoring system), and
administering said treatment to said patient if his/her liver scores at least F2.

The application also encompasses the use of at least one A2M peptide of the invention to in vivo produce an antibody or antibody fragment of the application. Said A2M peptide is then used as antigen, and may be linked to an immunogenicity carrier such as KLH or BSA. The application therefore encompasses the use of at least one A2M peptide of the invention, for its in vivo injection in an immunocompetent non-human animal and/or for the in vivo production of antibodies or antibody fragments. The application also encompasses any non-human immunocompetent animal (e.g., a rabbit), the body of which comprises at least one polypeptide of the application and/or at least one peptide of the application.

The application also encompasses the use of at least one A2M peptide of the invention to in vitro produce an antibody or antibody fragment of the application, e.g., by recombinant production.

The application also encompasses the use of at least one A2M peptide of the invention to screen antibodies or antibody fragments, more particularly to identify and/or isolate an antibody or antibody fragment of the application. Said peptide is then used as an affinity ligand, and may then e.g., be linked to a protein, polypeptide or peptide, which can bind solid support (such as a glass support or a plastic support e.g., a polystyrene solid support), for example to BSA.

The term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the invention.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### EXAMPLES

### Example 1

Materials and Methods

Serum Samples

Serum samples were collected at Beaujon Hospital (Paris, France) from 21 HCV-infected patients who were not undergoing treatment. The study was approved by the local ethics committee and was conformed to the 1975 Declaration of Helsinki. All patients gave written informed consent.

The degree of hepatic fibrosis in each HCV-infected patient was determined using the Metavir scoring scale. Nine patients had mild fibrosis (F1), ten patients had moderate fibrosis (F2), one patient had severe fibrosis (F3) and one patient had cirrhosis (F4). Only the serum samples from patients with F1 and F2 liver fibrosis (total n=19) were used for the differential study so as to focus on the identification of markers that might differentiate between F1 and F2 fibrosis; their clinical characteristics are listed in Table 1.

TABLE 1
Clinical parameters and total A2M concentration in serum of the
subjects under study. Results are expressed as medians (range).

| | Total | F1 HCV | F2 HCV |
|---|---|---|---|
| Patients (n) | 19 | 9 | 10 |
| Gender (M/F) | 10/9 | 2/7 | 8/2 |
| Age (years) | 53 (29-72) | 51 (29-66) | 53 (38-72) |
| BMI (kg/m²) | 24.4 (19.8-30.9) | 23.8 (19.8-29.7) | 24.7 (21.9-30.9) |
| total A2M | 2.97 (1.44-4.24) | 2.20 (1.50-3.49) | 3.71 (1.44-4.24) |
| (mg/mL) | | | |

Serum Fractionation

The AURUM® system (Bio-Rad, Hercules, Calif.) allows the depletion of the two most abundant proteins in serum (i.e., albumin and IgG) by using cross-linked agarose beads with covalently attached CIBACRON BLUE F3GA™ dye and protein A. Sixty microlitres of each crude serum sample were added onto the mini-column and depleted according to the manufacturer's instructions. Eluted, depleted samples were stored at −80° C. until analysis.

Protein Assay

The total protein content of each depleted serum sample was quantified using the QUICK START BRADFORD PROTEIN ASSAY KIT 2® (Bio-Rad) according to the manufacturer's instructions with bovine serum albumin (BSA) as standard (Bio-Rad).

One Dimensional Gel Electrophoresis (1-DE)

Ten micrograms of proteins from each depleted serum sample were diluted in Laemmli sample buffer (Bio-Rad) in which 5% 2-β-mercaptoethanol had been added. Samples were incubated at 95° C. for 10 min and ran through 12% Tris-HCl/SDS-PAGE gels. Gels were stained with COOMASSIE® Brilliant Blue R250. The signal intensity of each band was quantified with a GS-800 Densitometer (Bio-Rad) and the QUANTITY ONE 1D-ANALYSIS® software (Bio-Rad).

Two Dimensional Gel Electrophoresis (2-DE)

All depleted serum samples were treated with the ReadyPrep™ 2-DE Clean-up kit (Bio-Rad) before 2-DE analysis to remove impurities such as nucleic acids, lipids and salts. One hundred twenty or sixty micrograms of proteins from each sample were solubilised in 200 μL rehydration buffer (7 M Urea, 2 M Thiourea, 4% CHAPS, 15 mM DTT, 2 mM TBP, 0.2% or 0.4% Ampholytes, Bromophenol Blue) and loaded onto respectively 11 cm, pH 3-10 or pH 5.5-6.7 IPG strips (Bio-Rad). Strips were passively re-hydrated at 20° C. for 12 h. Proteins were isoelectrically focused at 250 V for 20 min (pH 3-10) or 15 min (pH 5.5-6.7), then at 8,000 V for 150 min (pH 3-10) or 60 min (pH 5.5-6.7) and finally maintained at 8,000 V for a total of 25,000 Vh/gel (pH 3-10) or 35,000 Vh/gel (pH 5.5-6.7). Strips from the first dimension separation were equilibrated in 2 mL reduction buffer (50 mM Tris pH 6.8, 6M Urea, 30% Glycerol, 2% SDS, 65 mM DTT) for 15 min and then in 2 mL alkylation buffer (50 mM Tris pH 8.8, 6M Urea, 30% Glycerol, 2% SDS, 81 mM Iodoacetamide) for 15 min. Strips were rinsed in 1× TGS running buffer (10× Tris/Glycine/SDS, Bio-Rad) and then loaded onto 4-20% Tris-HCl Precast Criterion linear gradient gels (Bio-Rad) that were run at 80 V for 20 min, then at 150 V for 1 h and at 200 V for 15 min in the CRITERION™ DODECA™ Cell system (Bio-Rad). Finally, proteins were blotted onto nitrocellulose membranes at 25 V for 45 min using the Trans-Blot SD Semi-Dry Transfer Cell system (Bio-Rad).

Western Blot Analysis

After 2-DE separation, serum proteins were blotted onto nitrocellulose membranes that were blocked in 5% non-fat dried milk/Phosphate Saline Buffer (Dulbecco) overnight. They were probed with 1 μg/mL of anti-human A2M goat polyclonal antibody (R&D Systems, AF1938) and then with anti-goat IgG HRP conjugated secondary antibody (1/80 000 dilution) (Sigma, A5420). Antibody binding was detected with ECL Western Blotting Detection Reagents (GE Healthcare) and Kodak BioMax Light-2 Films (VWR) were used. For detection of the N-terminal and C-terminal regions of A2M , nitrocellulose membranes were probed respectively with 1 μg/mL of anti-human N-Ter A2M mouse monoclonal antibody (Abnova, H00000002-M03) and 1 μg/mL of anti-human C-Ter A2M rabbit polyclonal antibody (Abcam, ab58703). Then, membranes were probed respectively with an anti-mouse (1/80,000 dilution) (Sigma, A9044) and an anti-rabbit IgG HRP conjugated secondary antibody (1/80,000 dilution, Sigma, A9169).

Differential Image Analysis

The PROGENESIS SAMESPOT® software (Non Linear Dynamics, Quayside, UK) was used for spot detection, quantification and comparison. The 2-DE gels of the 19 serum samples (patients with F1 and F2 liver fibrosis) fractionated with AURUM® were aligned. After automatic matching of the spots, gels were manually inspected to detect matching errors. The intensity of each spot was normalized to the total intensity of all the valid spots in the whole gel. Spots in gels from patients with F1 and F2 liver fibrosis were considered to have a significantly different intensity based on the following fixed parameters: ANOVA <0.05 and median fold change >1.3.

Determination of the N-Terminal or C-Terminal Localization of the A2M 40 kDa Fragments

To determine whether the A2M 40 kDa fragments are N-terminal or C-terminal A2M fragments, an anti-human N-terminal A2M mouse monoclonal antibody (Abnova, H00000002-M03) and an anti-human C-terminal A2M rabbit polyclonal antibody (Abcam, ab58703) have been used. The sequence of the respective epitopes of these antibodies has been determined by the SPOT method [Franck 1992].

Overlapping synthetic peptides corresponding to the amino acid sequence of human A2M (Expasy accession number P01023) were prepared using the epitope mapping technique [Franck 1992]. Two series of membrane-bound peptides (overlapping 15-mer peptides with a frameshift of two residues) were synthesized according to the procedure described by Molina et al. 1996.

After synthesis, non-specific binding to the membranes was blocked by incubation with TBS-TWEEN® 20% saccharose, blocking buffer diluted 1/50 (Roche, Germany) at room temperature overnight, and then membranes were probed with anti-N-terminal or anti-C-terminal antibodies (1 μg/mL in TBS-Tween 20% Saccharose, blocking buffer diluted 1/50). Antibody binding (anti-mouse HRP conjugated, Sigma, A9044; or anti-rabbit HRP conjugated, Sigma, A9169; 1/80 000 dilution) was revealed by incubation (at room temperature for 60 min) with ECL (GE Healthcare, RPN2106).

In-Gel Digestion

The 40 kDa A2M region from a 2-DE gel stained with COOMASSIE® Brilliant Blue R250 (150 μg of commercially available total A2M purified from human plasma; Sigma, M6159) was manually cut into pieces of about 2 mm² and transferred to 2 mL EPPENDORF® tubes. Pieces of gel were first washed with 1 mL of water and then 1 mL of 25 mM NH₄HCO₃. De-staining was performed twice in the presence of 1 mL of 50% acetonitrile in 25 mM NH₄HCO₃. Pieces of gel were dehydrated twice by 1 mL of 100% CH₃CN, and finally dried at room temperature.

Tryptic Digestion:

Thirty microliters of a trypsin solution (Sequencing Grade Modified Trypsin, Promega, Madison, USA), at a concentration of 0.0125 μg/μL in 25 mM NH₄HCO₃, were added to every gel pieces and were kept for 15 min on ice. Forty microlitres of 25 mM NH₄HCO₃ were added, and the samples were kept another 15 min at room temperature. Digestion was performed overnight at 37° C.

Endoproteinase Glu-C Digestion:

Ten microliters of a Endoproteinase Glu-C solution (Sequencing Grade from Staphylococcus aureus V8, Roche, Germany), at a concentration of 0.1 μg/μL in 25 mM NH₄HCO₃, were added to every gel pieces that were kept for 15 min on ice. Sixty microlitres of 25 mM NH₄HCO₃ were added, and the samples were kept another 15 min at room temperature. Digestion was performed overnight at 25° C.

After digestion, peptides were extracted by addition of 200 μL of 75% acetonitrile in water 2% formic acid. Digests were sonicated in an ultrasonic bath for 10 min and supernatants were transferred into 0.5 mL EPPENDORF® tubes then dried in a vacuum centrifuge. The peptides were suspended in 10 μL 2% formic acid, and moved in HPLC polypropylene tubes.

Mass Spectrometry

The protein digests were analyzed using 2 different liquid chromatography—tandem mass spectrometry (LC-MS/MS) configurations: a High Capacity ion trap mass spectrometer (Esquire HCT; Bruker Daltonik GmbH, Bremen, Germany) interfaced with a nano-HPLC Chip-Cube system (Agilent Technologies, Santa Clara, USA) and a QTOF mass spectrometer (Maxis; Bruker Daltonik GmbH, Bremen, Germany) interfaced with a nano-HPLC Ultimate 3000 (Dionex, Sunnyvale, USA).

Chip-Cube—HCT Configuration:

The chips contained both the pre-column and the column (Zorbax 300SB-C18; Agilent Technologies, Santa Clara, USA). Samples were first loaded onto the 4 mm enrichment cartridge at a flow rate of 4 μL/min using solvent A (0.1% formic acid in water, v/v). After pre-concentration, peptides were separated on the column (75 μm diameter, 43 mm length) at a flow rate of 0.3 μL/min using a 30 min linear gradient from 3% to 45% solvent B (0.1% formic add, 90% acetonitrile in water, v/v/v), and eluted into the mass spectrometer. A capillary voltage of 1.8-2.1 kV in the positive ion mode was used together with a dry gas flow rate of 4.5 L/min at 250° C. A first full-scan mass spectrum was measured in the 310 m/z to 1,800 m/z range, followed by a second scan at higher resolution to measure precisely the mass of the three major ions in the previous scan. Finally, a third scan was performed to acquire the collision-induced MS/MS spectra of the selected ions.

Ultimate 3000—Maxis Configuration:

The protein digests were analyzed using a QTOF mass spectrometer (Maxis; Bruker Daltonik GmbH, Bremen, Germany), interfaced with a nano-HPLC Ultimate 3000 (Dionex, Sunnyvale, USA). Samples were first loaded onto the pre-column (C18 PepMap100, 300 μm×5 mm, 5 μm, 100 A, Dionex) at a flow rate of 20 μL/min for 3 min with solvent A (0.1% formic acid, 2% acetonitrile in water, v/v/v). After pre-concentration, peptides were separated on the reversed-phase column (C18 PepMap100, 75 μm×250 mm, 3 μm, 100 A, Dionex) at a flow rate of 0.3 μL/min using a 69 min linear gradient from 8% to 27% solvent B (0.1% formic acid, 90% acetonitrile in water, v/v/v), and eluted into the mass spectrometer. The instrument was operated in the positive on mode and the nano-ESI source parameters are: a capillary voltage of 5000 V, a nebulization gas pressure of 0.4 bars and a dry gas flow rate of 4 L/min at 140° C. After an initial MS scan at 2 Hz over the mass range of 50-2,200 Th, the five most intense precursors were fragmented by collision-induced dissociation.

Mass Spectrometry Data Analysis

The tandem mass spectrometry (MS/MS) raw data obtained were analyzed using Data Analysis software (Bruker Daltonik GmbH, Bremen, Germany) to generate the peak lists. The UniProtKB/Swiss-Prot (release 20100121) was queried locally using the Mascot search engine (v. 2.2.04; Matrix Science, London, U.K.) and with the following parameters: Human for the taxonomy, semi-trypsin or semi-endoGluV8 as enzyme, 2 missed cleavages allowed, carbamidomethylation of Cystein as fixed modification, oxidation of Methionine as variable modification, 0.1 Da mass accuracy in MS and 0.3 Da in MS/MS for HCT experiment vs. 10 pmm in MS and 0.05 Da in MS/MS for Maxis experiments. Under these conditions, peptides with a p-value <0.05 were automatically validated. Peptides with lower scores were manually validated when fragmentation displayed at least 4 y-ions with 3 of them consecutive within the 10 most intense peaks.

N-Terminal Sequencing of the 40 kDa A2M Fragments

10 and 100 μg of commercially available human A2M (Sigma) were separated on Tris-HCl 12% SDS-PAGE gels. Proteins were blotted by liquid transfer (10 mM CAPS pH11, 10% methanol) onto a PVDF membrane and stained with a solution made of 0.1% Brilliant Blue R250, 40% methanol, 30% acetic acid. The three distinct bands were well-visualized after staining.

The bands corresponding to the 40 kDa A2M fragments were excised and stored at −20° C. until analysis at the IBS facility (Grenoble, France).

Amino acid sequencing was carried out following the Edman degradation method and using a gas-phase sequencer (Applied Biosystems, model 492). The phenylthiohydantoin amino acid derivatives generated during each sequence cycle were identified on-line with an Applied Biosystems Model 140C HPLC system using the Applied Biosystems 610A protein sequencer data analyzer (software version 2.1). Procedures and reagents were as recommended by the manufacturer.

Statistical Analysis

Variables were summarized as median values (±standard errors) and compared using the Wilcoxon test or one-way analysis of variance (ANOVA). The p values were calculated using the Progenesis Samespot software and the KaleidaGraph 4.0 software.

Results

2-DE Determination of A2M Fragments in Serum from HCV-Infected Patients

Serum samples from 19 HCV-infected patients with F1 and F2 liver fibrosis were pre-fractionated to specifically deplete two high-abundance proteins (albumin and IgG) (see FIG. 1A for a schematic description of the experimental procedure).

A sample from a patient with F2 fibrosis was used to optimize the 2-DE separation of serum protein. Briefly, the depleted sample was first separated using wide range IPG strips (pH 3-10) and the A2M fragments were visualized by western blot analysis using a polyclonal anti-A2M antibody. The full length monomeric A2M form (180 kDa), the thioester-cleaved N- and C-terminal A2M fragments (about 110 and 56 kDa respectively) and the bait region-cleaved N- and C-terminal A2M fragments (about 75 and 85 kDa respectively) were clearly detected (FIG. 1 B). Furthermore, new A2M fragments of about 40 kDa were detected as several distinct spots.

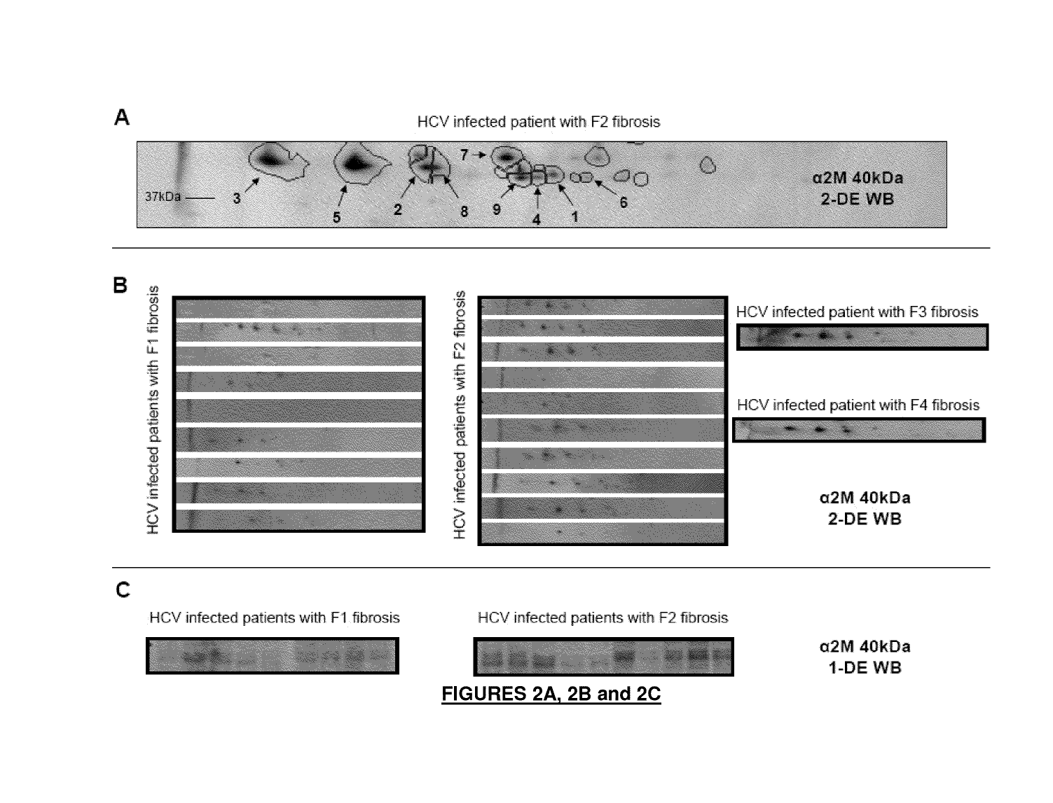

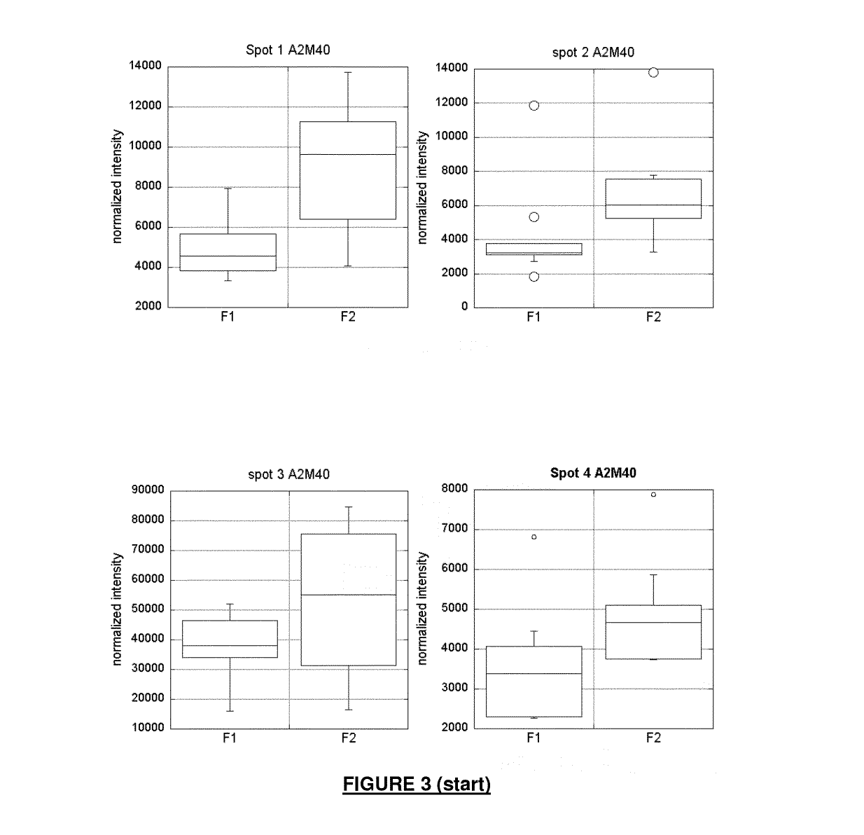

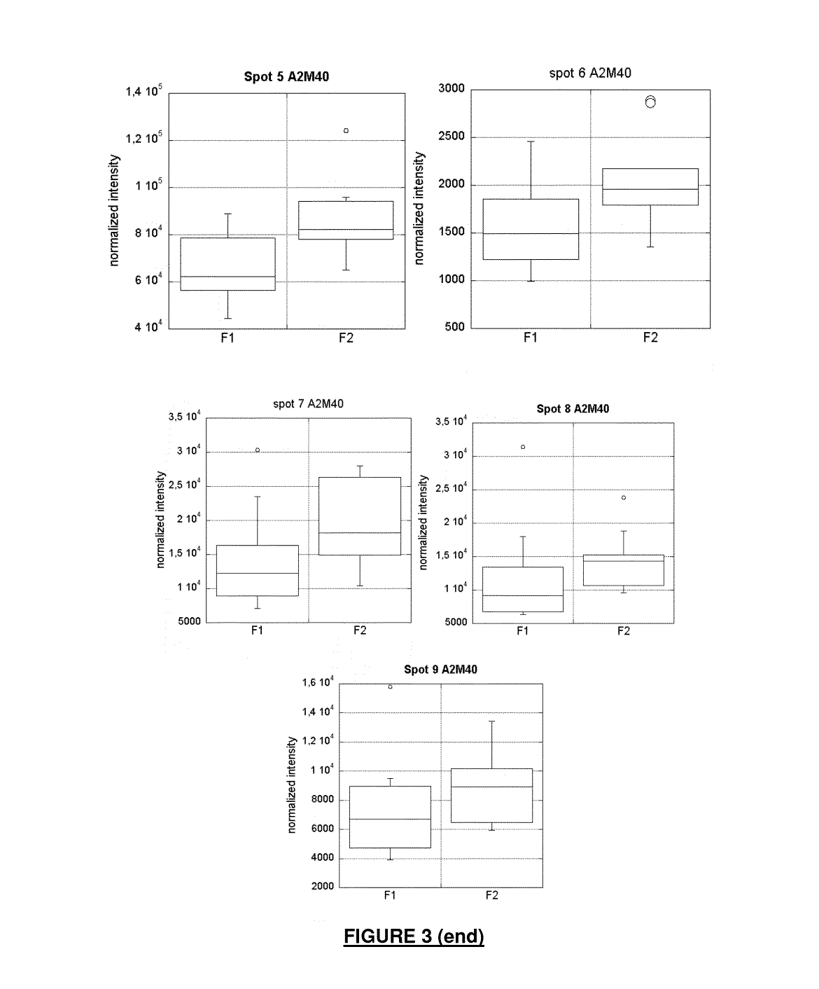

Then, the 2-DE separation of human serum A2M was optimized by using narrow range IPG strips (pH 5.5-6.7) to be closer to the isoelectric point (pi) of A2M (5.98) in order to better investigate these new 40 kDa A2M fragments. More resolved protein spots corresponding to the A2M monomer and to all the previously described fragments could be observed (FIG. 1B). More particularly, 18 spots in the region of the 40 kDa A2M fragments were detected (FIG. 1 B; FIG. 2A).

To the best of our knowledge, the A2M fragments of about 40 kDa are novel A2M fragments.

They are differently abundant in F1 and F2 patients, i.e., they are present at a significantly higher level in the F2 patients compared to the F1 patients.

Please note that the thioester-cleaved C-terminal A2M fragment of about 56 kDa, from which said new 40 kDa fragments may derive by cleavage, is not differently abundant in the F1 and F2 patients: at such early fibrosis stages, the 56 kDa fragment is not discriminative, contrary to the 40 kDa fragments.

2-DE and 1-DE Differential Analysis of the 40 kDa A2M Fragments in Serum Samples from HCV-Infected Patients with F1 and F2 Liver Fibrosis

To analyze the diagnostic potential of the new A2M fragments found in the 40 kDa region to differentiate HCV-infected patients with mild (F1 patients) from those with moderate fibrosis (F2 patients), a 2-DE differential analysis was performed using the 19 depleted serum samples from F1 (n=9) and F2 (n=10) patients and narrow range IPG strips (pH range 5.5-6.7) (one gel for each patient). The different A2M fragments were immunodetected with the polyclonal anti-A2M antibody. Nine of the 18 A2M spots in the 40 kDa region were found to be significantly more intense in the samples from F2 patients (FIGS. 2A and 2B). The intensity of the nine spots showed a fold change >1.30 between F1 and F2 samples and five of them had a p-value <0.05 (Table 2 and FIG. 3).

TABLE 2
2-DE spot and 1-DE band intensity in samples from HCV-positive
patients with F1 and F2 liver fibrosis (Metavir fibrosis stages).
Fold changes are expressed as medians (range).

F1 (n = 9) vs
F2 (n = 10)

| A2M 40 kDa | p-value | Fold Change |
|---|---|---|
| 2-DE spot 1 | 0.003 | 2.11 |
| 2-DE spot 2 | 0.022 | 1.87 |
| 2-DE spot 3 | 0.198 | 1.44 |
| 2-DE spot 4 | 0.038 | 1.38 |
| 2-DE spot 5 | 0.015 | 1.28 |
| 2-DE spot 6 | 0.038 | 1.31 |
| 2-DE spot 7 | 0.108 | 1.48 |
| 2-DE spot 8 | 0.159 | 1.57 |
| 2-DE spot 9 | 0.173 | 1.33 |
| 2-DE 9-spots | <0.05 | 1.55 |
| 1-DE 3 bands | <0.05 | 1.89 |

The region in which the nine spots were localized presented a fold change of 1.55 and a p-value <0.05. The other nine A2M spots identified in the 40 kDa region (FIG. 2A) were not differentially abundant in serum samples from F1 and F2 patients and they were also not the strongest spots of this region. Overall, the region including all the 18 spots corresponding to the A2M 40 kDa fragments presented a fold change of 1.26 between gels from F1 and F2 patients. By analyzing two more sera from HCV infected patients with F3 and F4 stages of fibrosis, we found that the A2M fragments in the 40 kDa region were more concentrated than in sera from F2 and F1 patients (FIG. 2B).

The depleted serum samples from F1 and F2 patients were also used to perform a 1-DE differential analysis. Immuno-detection of the different A2M fragments by the polyclonal anti-A2M antibody visualized two major and one minor distinct 40 kDa bands that were differentially abundant in F1 and F2 patients with a fold change of 1.89 and a p-value <0.05 (FIG. 2C and Table 2).

Finally, the possible correlations between gender, age and total A2M concentration (Table 1) and the signal intensity of the A2M 40 kDa fragments were investigated. Gender, age > or <45 years did not show any significant correlation with the signal intensity of the nine significant spots corresponding to A2M 40 kDa fragments (all p-values >0.05). However, total A2M concentration (> or <2.97 mg/mL, median concentration of total A2M in F1 and F2 patients) was correlated to the signal intensity for the spots 1, 7, 8 and 9 with respectively, p-values of 0.013, <0.001, 0.035 and 0.017. The spots 7, 8 and 9 didn't show significant p-values in the A2M 40 kDa fragment analysis but only significant fold changes (Table 2). The strongest spots in the 40 kDa A2M region (spots 1 to 6) were differentially abundant in HCV-positive patients with F1 and F2 liver fibrosis with high fold change and significant p-value (<0.05).

Determination of the N-Terminal or C-Terminal Localization of the A2M 40 kDa Fragments

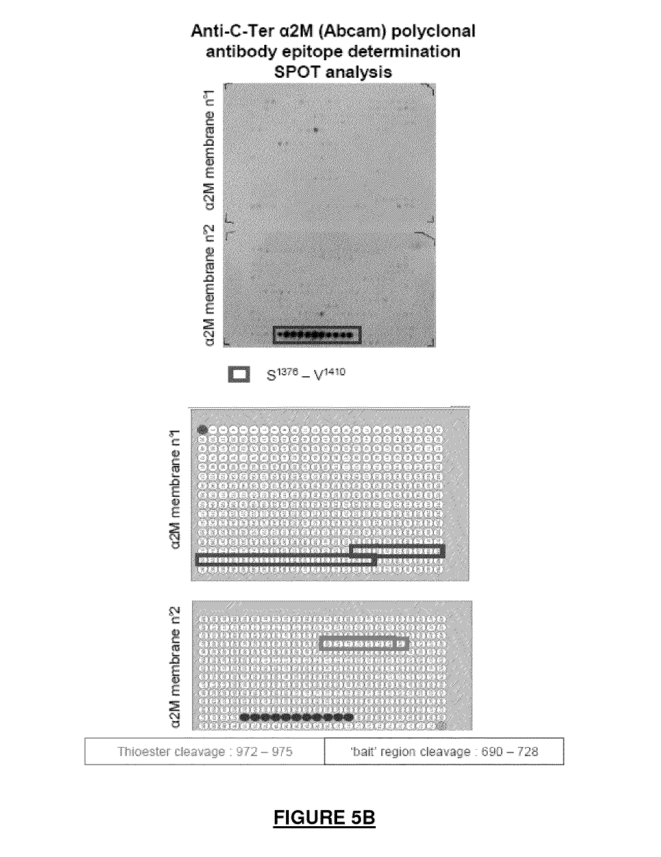

In order to determine the localization of the A2M 40 kDa fragments in the A2M protein sequence, two antibodies directed against A2M epitopes localized respectively in the C- and N-terminal regions of the protein were used. First, the epitopes of these antibodies were determined using the epitope mapping technique. The epitope of the anti-human C-terminal A2M rabbit polyclonal antibody (Abcam) corresponded to amino acids 1,376-1,410 and the epitope of the anti-human N-terminal A2M mouse monoclonal antibody (Abnova) to amino acids 662-679 (cf. FIGS. 5A and 5B). Then, the two anti-A2M antibodies were used to assess the amount of the different A2M fragments by western blotting in a 2-DE gel of a serum sample from a HCV-infected patient with F2 liver fibrosis (FIG. 4A). The C-terminal antibody detected the A2M 40 kDa fragments, whereas the N-terminal antibody did not. These results show that 40 kDa fragments are located in the C-terminal domain of human A2M. As expected, the already characterized A2M N-terminal and C-terminal fragments (i.e., the 105 kDa N-ter thioester-cleaved and the 75 kDa N-ter bait-cleaved fragments; the 56 kDa C-ter thioester-cleaved and the 85 kDa C-ter bait-cleaved fragments) were detected by antibodies in accordance with the epitope exposed (FIGS. 4A and 4B).

Determination of the Cleavage Sites Generating the A2M 40 kDa Fragments

To characterize the A2M 40 kDa fragments and determine the cleavage sites, which may generate the corresponding fragments, a LC-MS/MS approach was first employed. After trypsin digestion (cleavage after R or L) of the 2-DE gel section corresponding to the region containing the A2M 40 kDa fragments, the digested peptides were identified and their amino acid sequences obtained. The identified peptides selected based on their high frequency of appearance confirm a C-terminal localization of the A2M 40 kDa fragments (FIG. 4C). Three different peptides were identified (Table 3) that had different starting positions and that were likely to correspond to the N-terminus of the A2M 40 kDa fragments. These peptides [1,083-SGSLLNNAIK (SEQ ID NO: 7); 1,084-GSLLNNAIK (SEQ ID NO: 5); and 1,085-SLLNNAIK (SEQ ID NO: 3)] were localized in the C-terminal region of human A2M, after the bait region and the thioester bond cleavage sites (FIGS. 4B and 4C). They were selected based on their high frequency of appearance in HCT1, HCT2 and Maxis analyses (n=3). A second digestion of the peptides using the Glu protease (cleavage after D or E) confirmed the biologic origin of the three previously identified peptides (Table 3).

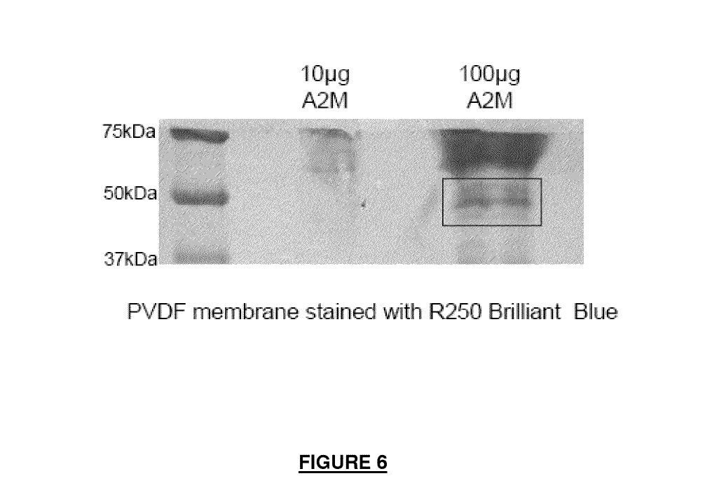

Finally, the N-terminal sequence of the A2M 40 kDa fragments was obtained by running 100 μg of A2M purified from human plasma on a 1-DE gel and then transferring it onto a PVDF membrane (cf. FIG. 6). The well-visualized bands corresponding to the A2M 40 kDa fragments were excised and their N-terminal sequences were determined. N-terminal sequencing confirmed the presence of the 1,085-SLLNNAIK peptide and also identified a new one [1,098-EVTLSAYITI (SEQ ID NO: 1)] (Table 3).

The 1,085 and 1,098 N-termini were confirmed by N-terminal sequencing. It was observed that the fragments starting at amino acid 1,085 or 1,098 are the most abundant of the four fragments. The fact that the 1,098 N-terminus was not found in the LC-MSMS experience certainly is likely due to the fact that this fragment is not well ionized, and therefore not detected using the mass spectrometry approach.

Moreover, the A2M Expasy tool (Gasteiger et al., 2005; software available from http://www.expasy.org/tools/protparam.html) calculated:
the theoretical length (390 amino acids for the 1,085-A2M 40 kDa fragment and 377 amino acids for the 1,098-A2M 40 kDa fragment),
the molecular weight (42.9 kDa and 41.5 kDa), and
the isoelectric point (6.02 and 6.18)

of the two A2M 40 kDa fragments starting at positions 1,085 and 1,098.

These values were in agreement with the results obtained by 2-DE and 1-DE analysis.

TABLE 3
Identification of the A2M-40 kDa fragment cleavage sites by LC-MS/MS and
N-terminal sequencing. The cleavage site sequences were identified using
the data gathered from three independent experiments and using different
apparatuses (HCT, High Capacity Trap ion mass spectrometer; ND, non
determined; X, Mascot identification in UniProtKB/Swiss-Prot with
p < 0.05; x, manual validation)

| | LC - MS/MS | | | | | |
|---|---|---|---|---|---|---|
| | Peptides identified | | | EndoGlu- | | |
| N Terminus | after enzymatic | Trypsin | | V8 | | N-terminal |
| A2M-40 kDa | digestion | HCT | Maxis | HCT | Maxis | sequencing |
| 1,085 - SLLNNAIK | SLLNNAIK/SLLNNAIKGGVEDE | X | X | X | X | 1,085 - SLLNNAIKXG |
| (SEQ ID NO: 2) | (SEQ ID NO: 2)/ | | | | | (SEQ ID NO: 16) |
| | (SEQ ID NO: 3) | | | | | |
| 1,084 - GSLLNNAIK | GSLLNNAIK/GSLLNNAIKGGVEDE | X | X | X | X | ND |
| (SEQ ID NO: 5) | (SEQ ID NO: 5)/ | | | | | |
| | (SEQ ID NO: 6) | | | | | |
| 1,083 - SGSLLNNAIK | SGSLLNNAIK/SGSLLNNAIKGGVEDE | X | X | ND | X | ND |
| (SEQ ID NO: 7) | (SEQ ID NO: 7)/ | | | | | |
| | (SEQ ID NO: 8) | | | | | |
| 1,098 - EVTLSAYITI | ND | ND | ND | ND | ND | 1,098 - EVTLSAYITI |
| (SEQ ID NO: 1) | | | | | | (SEQ ID NO: 1) |

Discussion

We have identified fragments of human A2M of about 40 kDa.

We demonstrate that these fragments are biomarkers of hepatic fibrosis, more particularly biomarkers of the early stages of hepatic fibrosis. A particularly advantageous feature is that they are biomarkers, which differentiate no/mild fibrosis (Metavir score of at most F1) from moderate/advanced/severe fibrosis (Metavir score of F2 or more). We found that the strongest spots in the 40 kDa A2M region were differentially abundant in HCV-positive patients with F1 and F2 liver fibrosis with high fold change and significant p-value (<0.05).

We found that, within the full length A2M protein, these new A2M fragments are localized in the C-terminal part of the protein sequence. We determined that the N-termini of these fragments correspond to amino acids 1,083, 1,084, 1,085 and 1,098 of full length A2M, respectively, the most abundant fragments being the 1,085- and 1,098-starting fragments.

Although human A2M is a well-known protein, it is believed that these new 40 kDa fragments localized in the C-terminal region of A2M have never been described before.

Human A2M monomer or thioester fragments have been reported in several proteomics studies, but, to the best of our knowledge, neither the 40 kDa fragments we have identified, nor any biological sub-fragment from the 56 kDa thioester-cleaved A2M fragment (i.e., any sub-fragment from said 56 kDa thioester-cleaved A2M fragment, which would have been observed to be naturally-occurring in the blood of a HCV-infected patient) have never been described before.

For example, when a posteriori analyzing the previously available data, it can be observed that, in a study on the primary structure of human A2M, Kristensen and coworkers described a 40 kDa fragment resulting from chemical cleavages with CNBr [Kristensen et al. 1984]. This 40 kDa fragment consisted of two CNBr fragments (CB21 and CB22) connected by an uncleaved Met-Thr bond and was located in the C-terminal part of A2M (CB21, residues 955-1,185 and CB22, residues 1,186-1,291). The calculated theoretical isoelectric point of the 40 kDa CNBr fragment was 7.85. Therefore it is too basic to correspond to the new 40 kDa biological fragments we identified. Furthermore, the N-terminal sequences do not correspond.

Furthermore, to the best of our knowledge, it has never been described before that the 40 kDa A2M fragments are markers of hepatic fibrosis, more particularly markers of the early stages of hepatic fibrosis, still more particularly markers, which differentiate no/mild fibrosis (Metavir score of at most F1) from moderate/advanced/severe fibrosis (Metavir score of F2 or more).

A2M serum concentrations were found to increase during hepatic fibrosis development [Naveau et al. 1994; Donnelly et al. 1983; Hiramatsu et al. 1976] and many non-invasive diagnostic tests have already included A2M as an independent predictor of the extent of fibrosis [Imbert-Bismut et al. 2001; Poynard et al. 2002; Patel et al. 2004; Cales et al. 2005].

For example, A2M total serum concentration has already been described to be fibrosis biomarker, but:
the A2M total concentration is the measure of several of the different A2M forms that are present in the patient's serum, without discriminating between these different forms: the A2M total concentration is the concentration of at least two elements from the group comprising the full length A2M monomer, the N-terminal and C-terminal thioester-cleaved A2M fragments, the N-terminal and C-terminal bait-cleaved A2M fragments; for example, when an anti-N-terminal A2M antibody has been used, the total A2M concentration is in fact the concentration of the A2M monomer+the N-terminal bait-cleaved A2M fragment of about 75 kDa+the N-terminal thioester-cleaved A2M fragment of about 105 kDa, and
the total A2M concentration has been described to be relevant for discriminating mild/moderate fibrosis (Metavir score of F1 or F2) from advanced/severe fibrosis (Metavir score of F3 or F4) [Imbert-Bismut et al. 2001; Poynard et al. 2002; Patel et al. 2004; Cales et al. 2005].

By comparison, it can be observed that:
our data do not relate to the A2M monomer, the bait-cleaved A2M fragments, the thioester-cleaved A2M fragments, but specifically relate to a particular group of A2M forms, i.e., the A2M fragments of about 40 kDa, and
our data demonstrate that these particular A2M fragments of about 40 kDa are present at a significantly higher level in the F2 patients compared to the F1 patients (i.e., our data's threshold is F2 versus F1, whereas, with the total A2M concentration, the threshold is F3 versus F2).

Some proteomic studies have confirmed the diagnostic potential of the A2M monomer (about 180 kDa) [Cheung et al. 2009; Ho et al. 2010].

Gangadharan and coworkers have shown that A2M thioester cleavage products are increased in cirrhotic patients (i.e., Metavir score of F4) and may give a more reliable indication of hepatic lesions [Gangadharan et al. 2007].

However, the thioester-cleaved C-terminal A2M fragment of about 56 kDa, from which said 40 kDa fragments could be considered to derive by cleavage, have not described to be a marker of hepatic fibrosis, and our data demonstrate that the F2 versus F1 differential abundance, which is observed for the A2M fragments of about 40 kDa, is not observed with the thioester-cleaved C-terminal A2M fragment of about 56 kDa.

Since our data indicate that the A2M 40 kDa fragments can be used to differentiate between F1 and F2 fibrosis, monoclonal antibodies against these A2M 40 kDa fragments can be developed. Such antibodies can be used to measure the concentrations of these new fragments in the serum of HCV-infected patients. Such antibodies can be used in a larger cohort of HCV-infected patients and/or with a cohort comprising more F3 and F4 patients and/or comprising F0 patients, to further illustrate the diagnostic potential of our 40 kDa A2M fragments.

Monomeric, homodimeric and homotetrameric members of the A2M family are widely distributed throughout the human species and share the ability to inhibit proteases, displaying different specificities and catalytic mechanisms. Several cell types including fibroblasts, macrophages, hepatocytes and dermal dendritic cells express a receptor for the A2M—protease complex for rapid internalization and intracellular degradation of the complex, whereas the receptor is recycled.

The new A2M 40 kDa biological fragments we identified may correspond to the A2M-proteinase complex binding domain that is generated by autolytic cleavage or nucleophilic reaction before complex internalization into the cell. Alternatively, the A2M 40 kDa fragments could be a degradation product of the 56 kDa C-terminal thioester fragment (FIG. 4B).

### Example 2

### Examples of Values of MW and of Isoelectric Point (pI)

TABLE 6

| | |
|---|---|
| A2M40 | number of |
| peptides | amino |

| start | stop | acids | MW (kDa) |
|---|---|---|---|
| 1081 | 1474 | 394 | 43.25 |
| 1083 | 1474 | 392 | 43.01 |
| 1084 | 1474 | 391 | 42.92 |
| 1085 | 1474 | 390 | 42.86 |
| 1098 | 1474 | 377 | 41.55 |
| 1123 | 1474 | 352 | 38.82 |
| 1081 | 1455 | 375 | 41.24 |
| 1083 | 1455 | 373 | 41.00 |
| 1084 | 1455 | 372 | 40.91 |
| 1085 | 1455 | 371 | 40.85 |
| 1098 | 1455 | 358 | 39.54 |
| 1123 | 1455 | 333 | 36.81 |

TABLE 7

| | |
|---|---|
| A2M40 | number of |
| peptides | amino |

| start | stop | acids | PI |
|---|---|---|---|
| 1081 | 1474 | 394 | 6.18 |
| 1083 | 1474 | 392 | 6.02 |
| 1084 | 1474 | 391 | 6.03 |
| 1085 | 1474 | 390 | 6.02 |
| 1098 | 1474 | 377 | 6.18 |
| 1123 | 1474 | 352 | 6.28 |
| 1081 | 1455 | 375 | 6.83 |
| 1083 | 1455 | 373 | 6.51 |
| 1084 | 1455 | 372 | 6.55 |
| 1085 | 1455 | 371 | 6.51 |
| 1098 | 1455 | 358 | 6.85 |
| 1123 | 1455 | 333 | 7.23 |

Start and stop positions are computed with respect to the full length human A2M sequence of SEQ ID NO: 9 (NM_{—}000014).

### Example 3

### Example of Antibody Production

CD-1 mice were immunized at 30 μg/injection with peptide 1,085-1,092 (SLLNNAIK; SEQ ID NO: 2) or peptide 1,098-1,104 (EVTLSAY; SEQ ID NO: 17) linked to a carrier protein (-Hx-GSGC-CONH2 for peptide 1,085-1,092; RSGRS-Diox-C-CONH2 for peptide 1,098-1,104) emulsified in adjuvant until antibody titer in sera was strong enough to perform a fusion. For each immunogen peptide, fusion was done between SP2/0 myeloma and splenocytes from immunized mouse. Hybridoma supernatants were screened through a funnel-shaped screening for antibodies capable of binding to recombinant or purified A2M-40 Kd and without binding to others A2M fragments. Screening format were ELISA, sandwich ELISA, western blot (or any other relevant formats). Primary screening post-fusion corresponding to 20 seeded 96 wells-plates was done by ELISA on A2M-40 Kd and counter-screening was done on full length A2M (FL-A2M). Up to 96 clones positive for A2M-40 Kd and negative on FL-A2M were selected. Confirmation of the 96 selected hybridomas was performed by ELISA on A2M-40 Kd and on immunogen peptide; counter-screening was done on FL-A2M and irrelevant peptides with Nter different from immunogen peptide. Up to 48 hybridoma positive on A2M-40 Kd and immunogen peptide, and negative on FL-A2M were selected. These 48 hybridomas were frozen and constituted the mother clones of the fusion. These clones were positive for the N-ter part of the A2M-40 Kd. Next step was the secondary screenings to choose up to 20 clones to be sub-cloned starting from the 48 mother hybridomas; screening formats were the following:
sandwich ELISA to identify antibodies that recognize free protein: anti-Cter A2M in capture/A2M-40 Kd/hybridoma/anti-mouse IgG-POD,
western-blot with native purified A2M (source Sigma that contains all forms of A2M including A2M-40 Kd) to identify antibodies that recognize native form,
epitope mapping to select only clones targeting Nter sequence of A2M-40 Kd.

For the epitope mapping of the antibodies produced with peptide 1,085-1,092:

(immunogen; SEQ ID NO: 2)
SLLNNAIK
(control other S in terminal position; SEQ ID NO: 18)
SSGSLLNNAIKGG
(control other S in terminal position; SEQ ID NO: 19)
SGSLLNNAIKGG
(control S85 non terminal; SEQ ID NO: 20)
GSLLNNAIKGG
(targeted sequence; SEQ ID NO: 21)
SLLNNAIKGG
(control without terminal S; SEQ ID NO: 22)
LLNNAIKGG
(control without terminal S; SEQ ID NO: 23)
ALLNNAIKGG
(Alascan to identify other potential essential aa; SEQ ID NO: 24)
SALNNAIKGG
(Alascan; SEQ ID NO: 25)
SLANNAIKGG
(Alascan; SEQ ID NO: 26)
SLLANAIKGG

For the epitope mapping of the antibodies produced with peptide 1,098-1,104:

(immunogen; SEQ ID NO: 17)
EVTLSAY
(control other E in terminal position; SEQ ID NO: 27)
EDEVTLSAYITI
(control E98 non terminal; SEQ ID NO: 28)
DEVTLSAYITI
(targeted sequence; SEQ ID NO: 29)
EVTLSAYITI
(control without terminal E; SEQ ID NO: 30)
VTLSAYITI
(control without terminal E; SEQ ID NO: 31)
AVTLSAYITI
(Alascan to identify other potential essential aa; SEQ ID NO: 32
EATLSAYITI
(Alascan; SEQ ID NO: 33)
EVALSAYITI
(Alascan; SEQ ID NO: 34)
EVTASAYITI
(Alascan; SEQ ID NO: 35)
EVTLAAYITI

Up to the 20 best clones were then sub-cloned and screened in ELISA, sELISA and WB. Then the 10 best sub-clones 1 were then sub-cloned again to ensure monoclonality. Screening of sub-clones 2 was done by ELISA and sELISA; the ten final best sub-clones-2 were then produced and purified.

### BIBLIOGRAPHIC REFERENCES

Ahmad W, Ijaz B, Gull S, Asad S, Khaliq S, Jahan S, Sarwar M T, Kausar H, Sumrin A, Shahid I, Hassan S, 2011. A brief review on molecular, genetic and imaging techniques for HCV fibrosis evaluation. Virol J 8: 53.

Harlow E and Lane E, 1988. Antibodies A Laboratory Manual. Cold Spring Harbor Laboratory press, Cold Spring Harbor, N.Y., U.S.A.

Cales P, Oberti F, Michalak S, Hubert-Fouchard I, Rousselet M C, Konate A, et al. A novel panel of blood markers to assess the degree of liver fibrosis. Hepatology. 2005; 42:1373-81.

Cales P, Boursier J, Oberti F, Hubert I, Gallois Y, Pousselet M C, Dib N, Moal V, Macchi L, Chevailler A, Michalak S, Hunault G, Chaigneau J, Sawadogo A and Lunel F, 2008. FibroMeters: a family of blood tests for liver fibrosis. Gastrcentérol Clinique et Biologique 32: 40-51.

Cales P, Boursier J, Bertrais S, Oberti F, Gallois Y, Fouchard-Hubert I, Dib N, Zarski J P and Rousselet M C, 2010. Optimization and robustness of blood tests for liver fibrosis and cirrhosis. Clin Biochem 43: 1315-1322.

Cheung K J, Tilleman K, Deforce D, Colle I, Van Vlierberghe H. The HCV serum proteome: a search for fibrosis protein markers. J Viral Hepat. 2009; 16:418-29.

Cole et al. 1983. Proc. Natl. Acad. Sci. USA 80:2026-2030

Cole et al. 1985. Monoclonal Antibodies And Cancer Therapy, Alan R. Liss, Inc., pp. 77-96

Donnelly P K, Shenton B K, Alomran A, Francis D M, Proud G, Taylor R M. The clinical relevance of alpha 2-macroglobulin. Ann NY Acad Sci. 1983; 421:382-7.

Frank R. Spot-Synthesis: an easy technique for the positionally addressable, parallel chemical synthesis on a membrane support. Tetrahedron. 1992; 48:9217-32.

Friedman S L. Evaluation of fibrosis and hepatitis C. Am J Med. 1999; 107:27S-30S.

Gangadharan B, Antrobus R, Dwek R A, Zitzmann N. Novel serum biomarker candidates for liver fibrosis in hepatitis C patients. Clin Chem. 2007; 53:1792-9.

Gasteiger E., Hoogland C., Gattiker A., Duvaud S., Wilkins M. R., Appel R. D., Bairoch A. Protein Identification and Analysis Tools on the ExPASy Server; (In) John M. Walker (ed): The Proteomics Protocols Handbook, Humana Press, 2005; pp. 571-607.

Guechot J, Lasnier E, Sturm N, Paris A, Zarski J P, 2010. Automation of the Hepascore and validation as a biochemical index of liver fibrosis in patients with chronic hepatitis C from the ANRS HC EP 23 Fibrostar cohort. Clin Chim Acta 411: 86-91.

Hiramatsu S, Kojima J, Okada T T, Inai S, Ohmori K. The serum protein profile in chronic hepatitis, cirrhosis and liver cancer. Acta Hepatogastroenterol (Stuttg). 1976; 23:177-82.

Ho A S, Cheng C C, Lee S C, Liu M L, Lee J Y, Wang W M, et al. Novel biomarkers predict liver fibrosis in hepatitis C patients: alpha 2 macroglobulin, vitamin D binding protein and apolipoprotein Al. J Biomed Sci. 2010; 17:58.

Imbert-Bismut F, Ratziu V, Pieroni L, Charlotte F, Benhamou Y, Poynard T., 2001. Biochemical markers of liver fibrosis in patients with hepatitis C virus infection: a prospective study. Lancet 357: 1069-75.

Köhler and Milstein 1975. Nature 256: 495-497.

Kosbor et al. 1983. Immunology Today 4: 72.

Kristensen T, Wierzbicki D M, Sottrup-Jensen L. Primary structure of human alpha 2-macroglobulin. IV. Primary structure of two large CNBr fragments, located in the COOH-terminal part and accounting for 337 residues. J Biol Chem. 1984; 259:8313-7.

Molina F, Laune D, Gougat C, Pau B, Granier C. Improved performances of spot multiple peptide synthesis. Pept Res. 1996; 9:151-5.

Naveau S, Poynard T, Benatar C, Bedossa P, Chaput J C, 1994. Alpha-2-macroglobulin and hepatic fibrosis. Diagnostic interest. Dig Dis Sci 39(11): 2426-2432.

Needleman and Wunsch 1970. Journal of Molecular Biology 48: 443-453.

Patel K, Gordon S C, Jacobson I, Hezode C, Oh E, Smith K M, et al. Evaluation of a panel of non-invasive serum markers to differentiate mild from moderate-to-advanced liver fibrosis in chronic hepatitis C patients. J Hepatol. 2004; 41:935-42.

Poynard T, Imbert-Bismut F, Ratziu V, Chevret S, Jardel C, Moussalli J, et al. Biochemical markers of liver fibrosis in patients infected by hepatitis C virus: longitudinal validation in a randomized trial. J Viral Hepat. 2002; 9:128-33.

Poynard T et al., 2004. Overview of diagnostic value of biochemical markers of liver fibrosis (FibroTest, HCV FibroSure) and necrosis (ActiTest) in patients with chronic hepatitis C. Comparative Hepatology 3: 8.

Shaheen A A, Wan A F, Myers R P, 2007. FibroTest and FibroScan for the prediction of hepatitis C-related fibrosis: a systematic review of diagnostic test accuracy. Am J Gastroenterol 102(11): 2589-2600.

U.S. Pat. No. 4,376,110

Zaman A, Rosen H R, Ingram K, Corless C L, Oh E and Smith K, 2007. Assessment of FIBROSpect II to detect hepatic fibrosis in chronic hepatitic C patients. Am J Med 120: 280, e289-214.

1. A polypeptide, the amino acid sequence of which is the sequence of a sub-fragment of the C-terminal thioester-cleaved fragment of human alpha-2-macroglobulin (A2M), wherein the molecular weight of said polypeptide is of 36 to 44 kDa, and wherein:
the first N-terminal amino acid of said polypeptide is an amino acid, which, in the full length sequence of said human A2M, is at a position 1,098 or 1,085 or 1,084 or 1,083, and
the last C-terminal amino acid of said polypeptide is an amino acid, which, in the full length sequence of said human A2M, is one of the last twenty C-terminal amino acids.

2. The polypeptide of claim 1, wherein said full length A2M amino acid sequence comprises 1,474 amino acids.

3. The polypeptide of claim 1, wherein the site of said thioester cleavage of said human A2M is at positions 972-975 and/or wherein said C-terminal thioester-cleaved fragment of said human A2M is of 56 kDa.

4. The polypeptide of claim 1, wherein said full length A2M amino acid sequence is the sequence of SEQ ID NO: 9, or a sequence which differs from the sequence of SEQ ID NO: 9 by at most five amino acid substitutions.

5. The polypeptide of claim 1, wherein said first N-terminal amino acid of said fragment is an amino acid, which, in the full length sequence of said A2M, is at position 1,098 or 1,085.

6. The polypeptide of claim 1, the amino acid sequence of which is SEQ ID NO: 12, SEQ ID NO: 13, SEQ ID NO: 14 or SEQ ID NO: 15.

7. The polypeptide of claim 1, which is of 39 to 43.5 kDa.

8. A peptide, the sequence of which is the sequence of a fragment from the polypeptide of claim 1, wherein:
the first N-terminal amino acid of said fragment is the first N-terminal amino acid of said polypeptide, and
said fragment comprises of at most 50 amino acids.

9. The peptide of claim 8, the sequence of which is one of SEQ ID NOs: 1-8, 17.

10. A compound comprising:
the polypeptide of claim 1, and of
one or more other structural components, which are linked to said polypeptide or peptide, wherein said other structural components are selected from the group consisting of His tags, BSA, and KLH.

11. A coding nucleic acid, the sequence of which comprises a sequence coding for the polypeptide of claim 1.

12. A vector, which comprises a coding nucleic acid inserted therein, wherein the expression product of said coding nucleic acid is the polypeptide of claim 1.

13. A genetically engineered host cell, which comprises the nucleic acid of claim 11.

14. An antibody or antibody fragment, which specifically binds to the N-terminal extremity of the polypeptide of claim 1, without binding to the full length human A2M protein.

15. The antibody of claim 14, which does not bind to the C-terminal thioester-cleaved fragment of said human A2M, and does not bind to the C-terminal bait-cleaved fragment of said human A2M.

16. The antibody of claim 14, which specifically binds to the N-terminal extremity of the polypeptide which comprises the non-covalently linked N-terminal alpha-amino group of said polypeptide.

17. A kit, comprising at least one antibody or antibody fragment of claim 14, and optionally at least one selected from:
at least one syringe, and/or
at least one tube, and/or
at least one resin having a selective binding affinity for albumin and/or immunoglobulin G, and/or
at least one protein denaturant.

18. An antibody or antibody fragment of claim 14, capable of being used for in vitro detecting of the polypeptide and/or for measuring concentration.

19. The antibody or antibody fragment of claim 14, capable of being used for in vitro detecting of hepatitis and/or liver fibrosis.

20. The antibody or antibody fragment of claim 14, capable of being used for in vitro scoring of a liver fibrosis stage and/or for determining whether a liver fibrosis is at a stage, which scores at most F1 or at a stage, which scores at least F2, in accordance with the Metavir scoring system.

21. The peptide of claim 8 capable of being used for in vivo injection in an immunocompetent non-human animal and/or for the in vivo or in vitro production of one or more antibodies or antibody fragments.

22. A non-human immunocompetent animal, the body of which comprises at least one polypeptide of claim 1.

23. The peptide of claim 8 capable of being used for in vitro screening of one or more antibodies or antibody fragments.


SEQUENCE LISTING (35 sequences)

SEQ ID NO: 1 - protein, 10 aa, Homo sapiens
EVTLSAYITI

SEQ ID NO: 2 - protein, 8 aa, Homo sapiens
SLLNNAIK

SEQ ID NO: 3 - protein, 14 aa, Homo sapiens
SLLNNAIKGGVEDE

SEQ ID NO: 4 - protein, 10 aa, Homo sapiens
SLLNNAIKGG

SEQ ID NO: 5 - protein, 9 aa, Homo sapiens
GSLLNNAIK

SEQ ID NO: 6 - protein, 15 aa, Homo sapiens
GSLLNNAIKGGVEDE

SEQ ID NO: 7 - protein, 10 aa, Homo sapiens
SGSLLNNAIK

SEQ ID NO: 8 - protein, 16 aa, Homo sapiens
SGSLLNNAIKGGVEDE

SEQ ID NO: 9 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 10 - protein, 1451 aa, Homo sapiens
SVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 11 - protein, 814 aa, Homo sapiens
TNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 12 - protein, 377 aa, Homo sapiens
EVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 13 - protein, 390 aa, Homo sapiens
SLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 14 - protein, 391 aa, Homo sapiens
GSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 15 - protein, 392 aa, Homo sapiens
SGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 16 - protein, 10 aa, Artificial Sequence
  N-terminal sequencing
SLLNNAIKXG

SEQ ID NO: 17 - protein, 7 aa, Homo sapiens
EVTLSAY

SEQ ID NO: 18 - protein, 13 aa, Artificial Sequence
  epitope mapping control
SSGSLLNNAIKGG

SEQ ID NO: 19 - protein, 12 aa, Artificial Sequence
  epitope mapping control
SGSLLNNAIKGG

SEQ ID NO: 20 - protein, 11 aa, Artificial Sequence
  epitope mapping control
GSLLNNAIKGG

SEQ ID NO: 21 - protein, 10 aa, Homo sapiens
SLLNNAIKGG

SEQ ID NO: 22 - protein, 9 aa, Artificial Sequence
  epitope mapping control
LLNNAIKGG

SEQ ID NO: 23 - protein, 10 aa, Artificial Sequence
  epitope mapping control
ALLNNAIKGG

SEQ ID NO: 24 - protein, 10 aa, Artificial Sequence
  epitope mapping control
SALNNAIKGG

SEQ ID NO: 25 - protein, 10 aa, Artificial Sequence
  epitope mapping control
SLANNAIKGG

SEQ ID NO: 26 - protein, 10 aa, Artificial Sequence
  epitope mapping control
SLLANAIKGG

SEQ ID NO: 27 - protein, 12 aa, Artificial Sequence
  epitope mapping control
EDEVTLSAYITI

SEQ ID NO: 28 - protein, 11 aa, Artificial Sequence
  epitope mapping control
DEVTLSAYITI

SEQ ID NO: 29 - protein, 10 aa, Homo sapiens
EVTLSAYITI

SEQ ID NO: 30 - protein, 9 aa, Artificial Sequence
  epitope mapping control
VTLSAYITI

SEQ ID NO: 31 - protein, 10 aa, Artificial Sequence
  epitope mapping control
AVTLSAYITI

SEQ ID NO: 32 - protein, 10 aa, Artificial Sequence
  epitope mapping control
EATLSAYITI

SEQ ID NO: 33 - protein, 10 aa, Artificial Sequence
  epitope mapping control
EVALSAYITI

SEQ ID NO: 34 - protein, 10 aa, Artificial Sequence
  epitope mapping control
EVTASAYITI

SEQ ID NO: 35 - protein, 10 aa, Artificial Sequence
  epitope mapping control
EVTLAAYITI